(19) United States
(12) Patent Application Publication
(10) Pub. No.: US 2010/0034871 A1
(43) Pub. Date: Feb. 11, 2010

(54) Center-Filled Chewing Gum Product For Dental Care

(76) Inventors: Rikke Mikkelsen, Vejle (DK); Tania Kjølhede Porsgaard, Brabrand (DK); Birgitte Kringelum Breindal, Kolding (DK)

Correspondence Address: ST. ONGE STEWARD JOHNSTON & REENS, LLC, 986 BEDFORD STREET, STAMFORD, CT, 06905-5619, US

(21) Appl. No.: 12/573,726

(22) Filed: Oct. 5, 2009

Related U.S. Application Data
(63) Continuation of application No. PCT/DK2007/000175, filed on Apr. 4, 2007.

(51) Int. Cl. A61K 9/68; B65D 75/00

(52) U.S. Cl. 424/440; 424/48; 229/87.07

(57) ABSTRACT

A center-filled chewing gum product includes functional agents and a chewing gum substance enclosing a center-filling, wherein the center-filling includes toothpaste and the functional agents include at least one anti-plaque agent.

### CROSS-REFERENCE TO RELATED APPLICATIONS

The present application is a continuation of pending International patent application PCT/DK2007/000175 filed on Apr. 4, 2007 which designates the United States and the content of which is incorporated herein by reference.

### FIELD OF THE INVENTION

The present invention relates to a center-filled chewing gum product which is effective for cleaning of teeth.

### BACKGROUND OF THE INVENTION

During the last several decades, the interest in cleaning of teeth has been growing. For most people, it is now of major interest to maintain clean and good-looking teeth as long as possible in life. Therefore, recommendations from dentists are followed as fully as possible, and most people are brushing their teeth at least twice every day.

In U.S. Pat. No. 6,524,023, a toothpaste dispensing device is disclosed, which in appearance resembles a center-filled chewing gum product. It is disclosed to prepare a product comprising a soft inner core surrounded by a stiffer outer layer. The soft inner core is disclosed as toothpaste, while the stiffer outer layer is disclosed as for example a chewing gum. The product is chewed to release the toothpaste and the chewing gum is spit out prior to brushing the teeth using a toothbrush.

However, brushing of teeth with a toothbrush is one of the reasons why problems such as abrasive damage to teeth and gingiva and exposure of dentine have become widespread today.

It is therefore an object of the present invention to provide a gentler dental care product so as to reduce the problems occurring as a result of conventional tooth brushing.

Moreover, it is an object of the invention to provide a center-filled chewing gum product, which is applicable as a replacement for a conventional toothbrush.

### SUMMARY OF THE INVENTION

The invention relates to a center-filled chewing gum product comprising functional agents and a chewing gum substance enclosing a center-filling, wherein

said center-filling comprises toothpaste and

said functional agents comprise at least one anti-plaque agent.

As used herein, the term “center-filling” refers to a filling in the center of the chewing gum product, although the filling does not necessarily need to be geometrically centered in the product, and the product does not necessarily need to be symmetric. However, in terms of production, it may be preferable to aim at substantial symmetry of the product and centering of the center-filling. For example, both storage stability of the center-filled chewing gum products and minimization of waste during production may be promoted by aiming at a rather unvarying wall-thickness when manufacturing the center-filled chewing gum product of the invention by an extrusion method.

As used herein, the words “center-filling” and “center-fill” are used interchangeably. Furthermore, the terms “centre” and “center” are used interchangeably.

As used herein, the term “toothpaste” includes dentifrices commonly known as toothpastes, dental creams and dental gels. Moreover, the term “toothpaste” as used herein refers to a composition comprising at least one liquid, at least one abrasive, at least one thickener, and at least one humectant. At least a part of the liquid may preferably be part of a pre-mixture with the humectant, for example in a preferred embodiment, liquid and humectant are added in the form of polyol syrup, such as maltitol syrup, sorbitol syrup, etc.

As used herein, the term “functional agent” is covering such terms as functional ingredients or components; active agents, ingredients or components; therapeutic agents, ingredients or components; and the like. The term “functional agents” is used herein as referring to compounds, ingredients, components, and agents having at least a dental care function.

As used herein the term “dental care” refers to oral care and particularly care of dental areas including gingiva, tongue and generally the teeth and mouth cavity, which may be treated when applying a conventional toothbrush.

According to the invention, a chewing gum product is provided, which may carry an adequate amount of toothpaste for one dental care treatment for one person and which when chewed releases anti-plaque agent and may provide dental care comparable with conventional tooth brushing. The anti-plaque agent is essential in order for the center-filled chewing gum product to be effective for toothbrush-replacement purposes.

The dental care center-filled chewing gum product according to the present invention may be used for treatment of teeth, mouth cavity and tongue to obtain dental care comparable to dental care obtained by a toothbrush. Although comparable, differences exist, and in fact considerable benefits may be obtained by applying a center-filled chewing gum product according to the invention instead of a toothbrush.

A particular advantage of applying a center-filled chewing gum product according to the present invention as compared to conventional brushing with a toothbrush is that the dental care obtained by the center-filled chewing gum product is considerably gentler to teeth and gingiva. Thus, the damages generally experienced as a result of tooth brushing may be reduced or avoided by replacing the conventional toothbrush with the center-filled chewing gum product according to the invention.

For some people, abrasive damage due to tooth brushing has become a severe problem, and their teeth have become sensitive. Nevertheless, their need for dental care has not been reduced and therefore they have been forced to continue brushing their teeth. However, during use of the center-filled chewing gum according to the invention, abrasive damage may be substantially avoided. Thus, the advantage of obtaining dental care without damaging the teeth or gingiva has been obtained. Therefore, applying the center-filled chewing gum according to the invention as a replacement for the toothbrush every time or at least every second, third or fourth time their teeth need cleaning, may reduce the continued abrasive damage experienced by the users.

Furthermore, application of the center-filled chewing gum product according to the invention on a regular basis as a replacement for a toothbrush may prevent the undesired effect, abrasive damage, which is often a result of subjecting the teeth to the bristles of a toothbrush. In other words, the positive dental care effects of conventional tooth brushing may be emulated and the negative effects may be avoided by use of the center-filled chewing gum product of the invention. In certain cases, it may also be a convenient dental care solution to apply the center-filled chewing gum product of the invention when a toothbrush is not available.

Thus, according to the present invention, a center-filled chewing gum product has been provided, which is suitable for use as replacement for a conventional toothbrush applied with toothpaste.

In an embodiment of the invention, the functional agents further comprise one or more agents selected from the group consisting of whitening agents, fresh-breath agents, anti-gingivitis agents, and re-mineralization agents, or any combination thereof.

The dental care obtained by the product according to the invention, and the emulation of the positive dental care effects usually attained by a toothbrush may be improved by each of the functional agents for providing fresh-breath, or whitening of the teeth, or fighting gingivitis, or promoting the re-mineralization process of the teeth and in particular of the enamel.

In an embodiment of the invention, the functional agents comprise at least one whitening agent, at least one fresh-breath agent, at least one anti-gingivitis agent, and at least one re-mineralization agent.

According to a preferred embodiment of the invention, anti-plaque agent(s), whitening agent(s), fresh-breath agent(s), anti-gingivitis agent(s) and re-mineralization agent(s) are all included in the center-filled chewing gum product and a product may be obtained, which is particularly effective in targeting a complete replacement of conventional tooth brushing.

In some embodiments of the invention, some functional agents may be applied for more than one purpose. An example for illustration may be the compound osteopontin, which is a functional agent, and which may serve as both re-mineralization agent, anti-gingivitis agent and anti-plaque agent.

In an embodiment of the invention, the functional agents further comprise at least one anti-calculus agent.

According to an embodiment of the invention, addition of anti-calculus agent in the center-filled chewing gum product may provide a product, which is more effective in fighting of calculus problems, and thereby emulation of a more thorough tooth brushing may be accomplished.

According to an embodiment of the invention, the anti-calculus agent may advantageously be incorporated in the center-filling, and the effectiveness of the anti-calculus agent may be obtained while avoiding any problems associated with incorporation of the anti-calculus agent in the chewing gum substance.

In an embodiment of the invention, the toothpaste comprises at least one anti-calculus agent.

According to an embodiment of the invention, the center-filled chewing gum product provides a way of incorporating anti-calculus agents in a chewing gum product containing further functional agents, which are not well suited to be in mixture with the anti-calculus agents. Accordingly, the anti-calculus agent and the other functional agents may be separated from each other by incorporating anti-calculus agents in the chewing gum substance and other functional agents in the center-filling, or reverse.

In an embodiment of the invention, the center-filled chewing gum product comprises said functional agents in an amount of 5% to 90%, preferably 10% to 70%, and most preferably 20% to 55% by weight.

According to a preferred embodiment of the invention, one or more functional agents may be included in the center-filling. An advantage hereby obtained may be a fairly immediate exposure of these functional agents to the teeth and oral cavity.

In an embodiment of the invention, said center-filling comprises one or more of said functional agents in a total amount of 10% to 90%, preferably 20% to 75% by weight of the center-filling.

In an embodiment of the invention, said center-filling comprises at least one anti-plaque agent, at least one whitening agent, at least one fresh-breath agent, at least one anti-gingivitis agent, and at least one re-mineralization agent.

According to an advantageous embodiment of the invention, functional agents serving different dental care purposes are included in the center-filling, whereby a combined action may be obtained almost immediately, and a consumer may gain the positive effects of the tooth brush emulation, even when the time period of treatment is shorter than the time recommended for tooth brushing and recommended for chewing gum chewing gum product according to the invention. As for functional agents requiring a longer time period for acting, it may more or less suffice that they are quickly delivered into the mouth, as long as the consumer does not wash his or her mouth right after using the center-filled chewing gum product.

Moreover, an advantage obtained according to the invention is that the center-filling may comprise functional agents, which are not suitable for being mixed into a chewing gum substance and thus are not applicable in a conventional non-filled chewing gum product. Desired functional agents, which need careful handling, may advantageously be added to the center-filling of the product according to the invention. Conversely, desired functional agents, which should be kept in a low moisture environment, may advantageously be added to the chewing gum substance.

In an embodiment of the invention, at least one of said functional agents is comprised in said toothpaste.

In an embodiment of the invention, said toothpaste comprises at least one anti-plaque agent, at least one whitening agent, at least one fresh-breath agent, at least one anti-gingivitis agent, and at least one re-mineralization agent.

In an embodiment of the invention, said toothpaste constitutes 10% to 100%, preferably 60% to 100%, and most preferably 90% to 100% of said center-filling.

In some embodiments of the invention, toothpaste may be purchased and mixed with further center-filling ingredients for providing specific tailor-made center-filling materials satisfying specific customized demands. For example, it may be the wish to apply a center-filling having a lower content of abrasive or a higher content of anti-plaque agent than what is present in the toothpaste at hand. It may therefore be preferred to mix such toothpaste with further ingredients to obtain a certain composition of the center-filling. In some cases, even though toothpaste having the right composition could be purchased, it may be chosen to adapt the toothpaste at hand, for example due to time- or cost-considerations.

In an embodiment of the invention, said toothpaste constitutes said center-filling.

When according to an embodiment of the invention the center-filling is constituted by toothpaste, the advantage is obtained that it is actually possible to make use of toothpaste, which has been produced by standard toothpaste manufacturing processes.

Especially when the toothpaste is incorporated with all the functional agents, which are desired for a certain center-filling, the advantage is obtained that such toothpaste may be applied directly as the center-filling of the center-filled chewing gum product according to the invention.

Thus, the advantage is obtained that the center-filling may be prepared in a process separated from the final manufacturing of the center-filled chewing gum products. Accordingly, in an embodiment of the invention, no functional agents need to be handled during the explicit process of manufacturing the center-filled chewing gum product. Thus, standard processes of preparing chewing gum products with center-fill may be utilized. Suitable processes of manufacturing such center-filled chewing gum products are for example described in WO2006/079338, WO2006/127277, U.S. Pat. No. 6,837,098, and US20060280834, hereby incorporated by reference.

Which functional agents are desired in the center-filling, such as a toothpaste, may depend on whether all of the chosen functional agents are suitable for being mixed or whether some of the functional agents may affect each other negatively. Encapsulation methods may be utilized for encapsulating vulnerable or aggressive functional agents, since such encapsulation may function as a protective barrier between the encapsulated agents and the surrounding mixture.

Furthermore, in an embodiment of the invention, the incorporation of all desired functional agents in the center-filling, such as a toothpaste, may facilitate that no functional agents need to be incorporated in the chewing gum substance. Conversely, in other embodiments of the invention, it may be preferred that some of the functional agents are included in the chewing gum substance while others are in the center-filling, since the center-filled chewing gum product according to the invention provides outstanding possibilities of separating different ingredients from each other for various reasons such as simply avoiding mixture of certain functional agents, or to obtain the advantage of different release patterns resulting from location in either the center-filling or the chewing gum substance.

In an embodiment of the invention, said chewing gum substance comprises one or more of said functional agents in a total amount of 5% to 90%, preferably 12% to 70%, and most preferably 30% to 60% by weight of the chewing gum substance.

In an embodiment of the invention, said chewing gum substance comprises whitening agent in an amount of 0.5% to 20%, preferably 1% to 15% by weight of the chewing gum substance.

In an embodiment of the invention, said chewing gum substance comprises fresh-breath agent in an amount of 0.5% to 10%, preferably 1% to 10% by weight of the chewing gum substance.

In an embodiment of the invention, said chewing gum substance comprises anti-plaque agents in an amount of 2% to 80%, preferably 10% to 60% by weight of the chewing gum substance.

In an embodiment of the invention, said chewing gum substance comprises anti-gingivitis agents in an amount of 0.5% to 20%, preferably 1% to 15% by weight of the chewing gum substance.

In an embodiment of the invention, said chewing gum substance comprises re-mineralization agents in an amount of 0.01% to 10%, preferably 0.1% to 10% by weight of the chewing gum substance.

In an embodiment of the invention, said chewing gum substance comprises anti-calculus agents in an amount of 0.5% to 30%, preferably 1% to 15% by weight of the chewing gum substance.

In an embodiment of the invention, said center-filling comprises one or more of said functional agents and said chewing gum substance comprises one or more of said functional agents.

In an embodiment of the invention, said center-filling and said chewing gum substance comprises functional agents of the same kind.

The “kind” of functional agents refers to both the specific ingredients and groups of functional agents. For example, the center-filling and the chewing gum substance may both comprise fresh-breath agents. Moreover, in an embodiment, the fresh-breath agent comprised in both center-filling and chewing gum substance could for example be green tea. Hereby, the effect of a certain kind of functional agent may be obtained on two fronts by delivering the agent by way of both center-filling and chewing gum substance.

In an embodiment of the invention, said center-filling and said chewing gum substance comprises functional agents of different kind.

According to an embodiment of the invention, different characteristics of the center-filling and the chewing gum substance may be advantageous for delivering different functional agents belonging to the same or different groups of functional agents.

In an embodiment of the invention, two different functional agents are separated in said center-filled chewing gum product by being located in said center-filling and said chewing gum substance, respectively.

The functional agents chosen to be in the center-filling may advantageously be chosen among the functional agents intended to be released quickly and almost immediately when the center-filled chewing gum product is chewed. Conversely, the functional agents intended to release slowly during use of the product may advantageously be located in the chewing gum substance.

In an embodiment of the invention, said toothpaste comprises at least one liquid, at least one humectant, at least one abrasive, and at least one thickener.

Typically, the liquid comprises water, and according to an embodiment of the invention, the liquid and humectant may be added to the toothpaste as a mixture such as syrup of a polyol.

In an embodiment of the invention, said at least one abrasive is selected from the group consisting of sodium metaphosphate, potassium metaphosphate, tricalcium phosphate, calcium phosphate dehydrate, anhydrous dicalcium phosphate, calcium pyrophosphate, magnesium orthophosphate, trimagnesium phosphate, calcium carbonate, baking soda, sodium hexametaphosphate, magnesium carbonate, magnesium silicate, titanium dioxide, zinc oxide, aluminum silicate, zirconium silicate, hydrated alumina, bentonite, hydrated silica, silica gel or colloidal silica, alkali metal aluminosilicate complexes and alumina, or any combination thereof.

According to an embodiment of the invention, the abrasives in the toothpaste may contribute in stabilization of the center-filling within the enclosing chewing gum substance. Moreover, an advantageous gentle abrasive effect may be obtained when chewing the center-filled chewing gum product, as the chewing action physically moves the chewing gum against the teeth bringing the abrasive component of the toothpaste into contact with the teeth while avoiding the abrasive damage caused by conventional brushing. The gentle abrasive effect obtained typically includes a cleaning, stain-removing and thus whitening effect.

In an embodiment of the invention, said at least one humectant is selected from the group consisting of glycerine, sorbitol, maltitol, xylitol, lactitol propylene glycol, polyethylene glycol, mannitol, polypropylene glycols, and mixtures thereof.

In an embodiment of the invention, said at least one thickener is selected from the group consisting of pectin, natural and synthetic gums, carboxylmethyl cellulose, sodium carboxymethylcellulose, hydroxyethylcarboxymethylcellulose, carrageenan, gum tragacanth, xanthan gum, guar gum, alginates, bentonite, other natural clays, synthetic inorganic clays, gums gelled with water or alkanols, and hydratable gums, irish moss, starch, hydroxybutylmethyl cellulose, hydroxypropylmethyl cellulose, hydroxyethyl cellulose, cellulose ethers, colloidal silica, karaya gum, gum arabic, or any combination thereof.

In an embodiment of the invention, said toothpaste comprises ingredients selected from the group consisting of surfactants, therapeutic agents, preservatives, and flavour.

Suitable surfactants may be selected among surfactants, which are anionic, nonionic, cationic, zwitterionic or amphoteric in nature. The therapeutic agents include the functional agents as defined according to the present invention.

In an embodiment of the invention, said toothpaste comprises a mixture of water and humectant, said mixture comprising 5-92% by weight, preferably 15-80% by weight, and most preferably 30-70% by weight of said toothpaste.

In preferred embodiments of the invention, the mixture of water and humectant is pre-mixed prior to addition into the toothpaste. In particularly advantageous embodiments of the invention, the mixture of water and humectant is a polyol syrup such as sorbitol syrup, mannitol syrup, maltitol syrup, etc.

A particular advantage is obtained by the center-filled chewing gum product according to the invention when functional agents are mixed into the toothpaste, and thus the center-filling. The water content of the toothpaste and center-filling provides that the functional agents may be dissolved in water and thereby be ready for acting on the teeth, dental areas, etc. of the consumer as soon as the product is chewed.

In an embodiment of the invention, said toothpaste comprises water in an amount of 2-90% by weight, preferably 5-80% by weight, and most preferably 10-70% by weight.

In an embodiment of the invention, said toothpaste comprises said at least one abrasive in an amount of at least 7%, preferably at least 10%, and most preferably at least 15% by weight.

In an embodiment of the invention, said at least one thickener comprise 0.05% to 10% by weight of said toothpaste.

In an embodiment of the invention, said toothpaste is edible. As chewing gum is usually associated with confectioneries, a consumer may also be incited to swallow the ingredients released from the chewing gum according to the present invention. It may therefore be preferred that the ingredients, including the toothpaste, are edible.

According to an advantageous embodiment of the invention, the center-filled chewing gum product comprising toothpaste in the center-filling may further comprise one or more abrasives included in the chewing gum substance. In this way, both the toothpaste center-filling and the chewing gum substance may provide sources for distribution of abrasive during chewing of the product. Hereby, a double effect is provided, and a gentle abrasive action may be achieved both in the beginning of and throughout a longer period of chewing.

In an embodiment of the invention, said whitening agents are selected from the group consisting of magnesium carbonate, calcium carbonate, calcium pyrophosphate, baking soda, sodium hexa-metaphosphate, magnesium silicate, silica, titanium dioxide, zinc oxide, and mixtures thereof.

In an embodiment of the invention, said fresh-breath agents are selected from the group consisting of chlorohexidine, hexetidine, delmopinol, zinc oxide, zinc silicate, zinc carbonate, zinc acetate, zinc phosphate, zinc stannate, zinc citrate, zinc, zinc oxalate, zinc stearate, zinc chloride, zinc sulfate, zinc nitrate, zinc compounds as a complexes, green tea, red tea, white tea, black tea, thyme, eucalyptus, and mixtures thereof.

Some of the most advantageous fresh-breath agents according to the invention include chlorohexidine (Merck Index, No. 2090), hexetidine (Merck Index, No. 4624), delmopinol, extracts obtained from tea (green tea, red tea, white tea and black tea), thyme, eucalyptus, zinc sources in the form of compounds with fresh breath properties (zinc oxide, zinc silicate, zinc carbonate, zinc acetate, zinc phosphate, zinc stannate, zinc citrate, zinc, zinc oxalate, zinc stearate, zinc chloride, zinc sulfate, zinc nitrate). Moreover, the zinc compounds may advantageously be present as a complex with a complexing agent such as polyethylenimine or ethylenediamine tetraacetic acid.

In an embodiment of the invention, said anti-plaque agents are selected from the group consisting of 2′,4,4′-trichloro-2-hydroxy-diphenyl ether (Triclosan), phenolic compounds (including phenol and its homologs, mono- and poly-alkyl and aromatic halo-phenol and their homologs), phenol, 2-isopropyl-5-methyl-phenol (Thymol), green tea and extracts thereof, white tea, red tea, coffee and extracts thereof, thyme, oregano, cranberry and extracts thereof, aronia, blueberry, eucalyptus, eucalyptus extract, polyphenoles extracted from berries and plants, zinc ions, copper ions, iron ions and other metal ions, delmopinol, xylitol, maltitol, immuglobuline-lysozyme, and mixtures thereof.

A suitable immuglobuline-lysozyme is e.g. Ig-lyt or IG-LY 4023 disclosed in patent WO 2004/060397 A1.

In an embodiment of the invention, said anti-gingivitis agents are selected from the group consisting of lactic acid bacteria (LAB), osteopontin (ONP), immuglobuline-lysozyme, aloe vera, chlorhexidine, green tea, phenolic compounds (including phenol and its homologs, mono- and poly-alkyl and aromatic halo-phenol and their homologs), polyphenoles extracted from berries and plants, and mixtures thereof.

In an embodiment of the invention, said re-mineralization agents are selected from the group consisting of dicalcium phosphate and any calcium ion thereof, sodium fluoro phosphate and any salt thereof, sodium fluoride and any ion from fluoride, nano-hydroxyapatite, Novamin®, calcium and phosphate-peptides, CPP-ACP, and mixtures thereof.

According to an embodiment of the invention, it is preferred to apply fluoride as one of the functional agents in order to provide the center-filled chewing gum product with this particularly effective agent both for re-mineralization purposes and as anti-caries agent.

In an embodiment of the invention, said functional agents comprise fluoride salts selected from the group consisting of sodium fluoride, potassium fluoride, sodium fluorosilicate, ammonium fluorosilicate, sodium fluorozirconate, sodium monofluorophosphate, or any combination thereof.

In an embodiment of the invention, said anti-calculus agents are selected from the group consisting of vitamin C, citric acid, and acetic acid, or any combination thereof.

In an embodiment of the invention, said functional agents comprise an anti-Streptococcus mutans agent.

Examples of functional agents suitable for counteracting Streptococcus mutans include but are not limited to triclosan, phenolic compounds, phenol, thymol, green tea and extracts thereof, white tea, red tea, coffee and extracts thereof, thyme, oregano, cranberry and extracts thereof, aronia, blueberry, eucalyptus, eucalyptus extract, polyphenoles extracted from berries and plants, zinc ions, copper ions, iron ions and other metal ions, delmopinol, xylitol, maltitol, immuglobuline-lysozyme, and mixtures thereof.

In an embodiment of the invention, said center-filling comprises at least a part of said fresh-breath agents.

In an embodiment of the invention, said center-filling comprises whitening agent in an amount of 0.1% to 50%, preferably 5% to 50%, and most preferably 10% to 50% by weight of the center-filling.

In an embodiment of the invention, said center-filing comprises fresh-breath agent in an amount of 0.1% to 50%, preferably 5% to 50%, and most preferably 10% to 50% by weight of the center-filling.

In an embodiment of the invention, said center-filing comprises anti-plaque agents in an amount of 10% to 90%, preferably 20% to 80%, and most preferably 30% to 70% by weight of the center-filling.

In an embodiment of the invention, said center-filing comprises anti-gingivitis agents in an amount of 0.1% to 50%, preferably 5% to 50%, and most preferably 10% to 50% by weight of the center-filling.

In an embodiment of the invention, said center-filing comprises re-mineralization agents in an amount of 0.1% to 50%, preferably 1% to 50%, and most preferably 10% to 50% by weight of the center-filling.

In an embodiment of the invention, said center-filing comprises anti-calculus agents in an amount of 0.1% to 50%, preferably 5% to 50%, and most preferably 10% to 50% by weight of the center-filling.

In an embodiment of the invention, said center-filling comprises in the range of 2 to 40%, preferably 5 to 20%, and most preferably 5 to 15% by weight of said center-filled chewing gum product.

In an embodiment of the invention, said center-filling comprises 1% to 95%, preferably 5% to 80% by weight of liquid.

According to an advantageous embodiment of the invention, the liquid-comprising center-filling is immobilized by way of its content of thickener. Thus, even with a high liquid content in the center-filling paste- or gel-like consistency can be maintained.

In an embodiment of the invention, said center-filling comprises a gelling agent.

In an embodiment of the invention, said center-filling comprises starch.

Gelling agents and starch may be utilized as thickeners of the center-filling according to the invention.

In an embodiment of the invention, said center-filling is at least partially solid at temperatures up to 5° C.

In further embodiments of the invention, said center-filling is at least partially solid at temperatures of up to 20° C. or 30° C.

An advantage may hereby be obtained that the center-filled chewing gum product may conveniently have a solid-like center-filling when stored at rather cold temperatures, such as in a refrigerator, or when stored at room temperature, whereas the user when taking the chewing gum into the mouth will experience a center-fill, which is liquid-, paste-, or gel-like. A solid-like consistency under storage may improve the keeping qualities of the center-filled chewing gum product considerably, as problems of leaking and dissolving of the chewing gum substance may hereby be avoided.

In an embodiment of the invention, said center-filling has a viscosity in the range of 0.6 to 200000, preferably 100 to 100000 mPa*s as measured at 40° C.

In an embodiment of the invention, said center-filling has a paste-like consistency.

The term “paste-like consistency” is considered at usual storage temperatures such as 15-25° C. and includes gel-like consistency. According to an embodiment of the invention, the viscosity of the paste-like center-filling may be in the range of 1000 to 200000 mPa*s as measured at 20° C.

In an embodiment of the invention, said center-filling is in the form of a paste, a gel, or any combination thereof.

According to an embodiment of the invention, the content of liquid in the center-filling may advantageously be rather low. Thereby a low mobility of functional agents may be obtained and moreover the keeping qualities and storage life of the final center-filled chewing gum product may be improved and prolonged.

In an embodiment of the invention, said center-filling comprises a mixture of solid parts and liquid parts.

In an embodiment of the invention, the center-filling and thus the toothpaste comprises a water activity which is adjusted in relation to the water activity in the chewing gum substance in order to avoid diffusion of water between the center-filling and the chewing gum substance. The water activity may advantageously be adjusted in order to avoid diffusion of water from the center-filling to the surrounding chewing gum substance, thus to avoid drying up of the center-filling and/or exaggerated moistening of the chewing gum substance. The water activity of the toothpaste and thus the center-filling may be recognized as a function of the ratio of water to humectant in the toothpaste formulation, as well as water to the content of thickeners, abrasives, and other dissolved species. By lowering the ratio of water to humectant, or by increasing the content of thickeners or other material which bind water, the water activity of the toothpaste composition can be lowered.

In an embodiment of the invention, said center-filling comprises 5% to 80% by weight of solid parts.

In an embodiment of the invention, said center-filling is substantially centered in the geometrical center of said center-filled chewing gum product.

In an embodiment of the invention, said center-filled chewing gum product is sugar free or sugarless.

In an embodiment of the invention, said center-filled chewing gum product comprises at least one bulk sweetener.

In an embodiment of the invention, said center-filled chewing gum product comprises at least one high-intensity sweetener.

In an embodiment of the invention, said center-filled chewing gum product comprises at least one foaming agent

The term “foaming agent” as used herein includes the term effervescent agent. Generally, a foaming agent may according to the invention be applied in order to serve the same purposes as are usually obtained when applying a foaming agent in toothpaste. According to an embodiment of the invention, the inclusion of a foaming agent may provide the center-filled chewing gum product with characteristics that improve the experience of teeth brushing during chewing of the center-filled chewing gum product of the invention, and thereby the emulation of tooth brushing is strengthened.

One of the advantages of the present invention is that a dental care product has been provided by which a consumer may obtain the effects of tooth brushing without actually brushing and thereby subjecting the teeth to abrasive damage. Thus, in the absence of a toothbrush it may be an advantage according to the present invention to include a foaming agent in the center-filled chewing gum product; the foam resulting from the foaming agent may help to carry the functional agents and distribute them throughout the mouth.

Preferably, only edible foaming agents are applied in the center-filled chewing gum product according to the invention.

In an embodiment of the invention, said foaming agent is a surfactant.

In an embodiment of the invention, said foaming agent comprises an effervescing system including edible acid and edible alkaline material.

In an embodiment of the invention, said center-filling is colored.

In an embodiment of the invention, said center-filling is colored in one, two, three, four, five, or six different colors.

In an embodiment of the invention, said center-filling is colored in stripes.

An advantageous embodiment of the invention may be obtained, when the center-filling comprises different colors appearing as a striped pattern. The different stripes may advantageously represent different dental care actions provided by the chewing gum product.

Conventional methods of manufacturing striped products during a continuous extrusion process are applicable according to the invention.

In an embodiment of the invention, said center-filling is colored in spots.

The spots may e.g. be created by way of colored beads in the center-filling. The beads may for example comprise flavour or functional agents and may appear as small colored particles in the center-filling. The particles, i.e. the spots, may appear in one or more colors and may represent dental care actions provided by the product.

The stripes or spots may e.g. be visible through a translucent chewing gum substance enclosing the center-filling, or they may simply be recognized subsequent to biting or cutting through the center-filled chewing gum product.

In an embodiment of the invention, different functional agents are located in different parts of said center-filling, said parts being in different colors.

In an embodiment of the invention, said chewing gum substance and said center-filling are colored differently.

According to some embodiments of the invention, a mean for enabling the user of the product to clearly distinguish the center-fill-part from the chewing gum-part may be to provide the chewing gum substance and center-filling with different colors. Hereby, an improved conception of toothpaste may be obtained, which may encourage the consumer to appreciate the center-filled chewing gum product as a tooth- and oral-care product. And less pleasant tastes resulting from functional agents may easier be accepted.

As a chewing gum by itself usually does not remind much of a toothbrush, it is preferred according to the invention to design the appearance of the chewing gum in a way that increases the conception of a toothbrush. Designing such parameters as shape, color, texture, and labelling may for example be applied. Moreover, as the present invention relates not just to a chewing gum but a chewing gum product provided with a center-fill, the products according to the invention are excellently suited for providing the consumer with an actual perception and experience of tooth brushing.

It is preferred according to some embodiments of the invention to design the appearance, color, texture, and consistency of the center-filling so as to resemble toothpaste. The intuitive perception by the consumer that the product is in fact a kind of toothbrush may hereby be improved.

In an embodiment of the invention, said chewing gum substance is colored in stripes or spots.

As the chewing gum substance is readily visible for the consumer, it may be preferred to apply colored patterns to the chewing gum substance. Patterns such as stripes or spots may be applied to signify different functionalities of the chewing gum product, preferably functionalities obtained by way of the applied functional agents according to the invention.

In an embodiment of the invention, said chewing gum substance is translucent.

As used herein, a “translucent” substance refers to a transparent or semi-transparent substance. Thus, a translucent substance permits light to pass, and the translucent chewing gum substance permits the center-filling inside to be visible, although the visual impression of the center-filling may typically be more or less blurred when seen through the chewing gum substance.

Suitable transparent or translucent chewing gum formulations are for example disclosed in the application PCT/DK2006/000233, which is hereby incorporated by reference.

A translucent chewing gum substance may according to an embodiment of the invention facilitate that the center-filling is visual for the consumer. This may provide particular advantages when the center-filling is colored, and the consumer may be enabled to visible recognize the presence of toothpaste within the center-filled chewing gum product prior to chewing. The pre-chewing recognition that the chewing gum product is a tooth- and oral-care product in line with a toothbrush may considerably enhance the expectations of a tooth brushing experience and thus the acceptance of tastes and sensations far from confectionery products.

In some embodiments of the invention, it is preferred to provide the center-filled chewing gum product with an outer coating for protecting the gum product and to provide the product with a consumer friendly appearance and sensation. Usually according to the invention, a translucent coating may be preferred when a translucent gum composition is selected.

In an embodiment of the invention, said center-filling is translucent.

In an embodiment of the invention, said center-filled chewing gum product comprises flavoring agent.

In an embodiment of the invention, said chewing gum substance and said center-filling comprises different flavoring agents.

In an embodiment of the invention, a barrier layer is located between said center-filling and said chewing gum substance.

In an embodiment of the invention, said barrier layer comprises one or more components selected from the group consisting of lipids, proteins, carbohydrates, synthetic elastomers, polymers, or combinations thereof.

According to some embodiments of the invention, a barrier layer may preferably be applied between the center-filling and the surrounding chewing gum substance. When the center-filling is made of a predominantly hydrophilic composition, the barrier layer may preferably be made of a hydrophobic substance, and reverse. A combination of hydrophilic and hydrophobic components in the barrier layer may often provide a preferred composition of the barrier layer.

In an embodiment of the invention, said barrier layer comprises one or more components selected from the group consisting of wax, fat, gelling hydrocolloid, agar, alginate, carrageenan, gluten, milk proteins, gelatin, starch, gellan gum, locust bean gum, pectin, pectinates, xanthan gum, and cellulose-ethers.

Disclosure of suitable barrier layers may be found in the application US20060263476, which is hereby incorporated by reference.

In an embodiment of the invention, one or more of said functional agents are encapsulated by one or more encapsulation agents.

According to an embodiment of the invention, it may be preferred to apply an encapsulation agent to encapsulate a functional agent and thereby obtain a sustained release of that functional agent.

In an embodiment of the invention, said encapsulation agents are selected from the group consisting of polyvinyl acetate, polyethylene, crosslinked polyvinyl pyrrolidone, polymethylmethacrylate, polylactidacid, polyhydroxyalkanoates, ethylcellulose, polyvinyl acetatephthalate, polyethylene glycol esters, methacrylicacid-co-methylmethacrylate, ethylene-vinylacetate (EVA) copolymer, fat, wax, natural resins, polyterpene resins, or combinations thereof.

In an embodiment of the invention, at least one of said encapsulation agents comprises a combination of natural resin and polyvinyl acetate.

According to an embodiment of the invention, an encapsulation agent comprising a mixture of natural resin and polyvinyl acetate provides a particularly advantageous delivery system.

In an embodiment of the invention, at least one of said encapsulation agents is included in the chewing gum substance.

In an embodiment of the invention, at least one of said encapsulation agents is included in the center-filling.

In an embodiment of the invention, said center-filled chewing gum product is coated by an outer coating.

Suitable coating types include hard coatings, film coatings and soft coatings.

Hard coatings comprise at least one polyol, which may be selected from the group consisting of sorbitol, maltitol, mannitol, xylitol, erythritol, lactitol, isomalt, and mixtures thereof. A film-coating may comprise at least one component selected from the group consisting of wax, cellulose derivatives, a modified starches, dextrins, gelatine, shellac, gum arabics, zein, vegetable gums, synthetic polymers, and mixtures thereof. A soft coating may e.g. comprise a mixture of a sweetening compound and a starch hydrolysate and may e.g. comprise a syrup of a polyol.

In an embodiment of the invention, said outer coating is colored.

In an embodiment of the invention, said chewing gum substance forms a wall enclosing said center-filling in substantially all directions.

In an embodiment of the invention, said wall has an average thickness in the range of about 0.5 to 10 mm, preferably about 1 to 5 mm, and most preferably 1.5 to 3 mm.

In an embodiment of the invention, said center-filled chewing gum product weighs in the range of 0.5 to 10 grams, preferably 1 to 7 grams, and most preferably 1.5 to 4 grams.

In an embodiment of the invention, said chewing gum piece is shaped as a pellet, chunk, stick, cushion, pastille, ball, pill, or sphere.

In an embodiment of the invention, said center-filling and said chewing gum substance have a volume-ratio in the range of 1:1 to 1:10.

In an embodiment of the invention, said center-filling and said chewing gum substance have a weight-ratio in the range of 1:1 to 1:10.

In an embodiment of the invention, said chewing gum substance comprises a gum base constituting 20 to 80%, preferably 30 to 60% by weight of the chewing gum substance.

In an embodiment of the invention, said chewing gum substance comprises at least one elastomer

In an embodiment of the invention, said gum base comprises high molecular weight elastomer of molecular weight about 300000 to 400000 g/mol in an amount of at most 3%, preferably at most 2% by weight of the chewing gum substance.

In an embodiment of the invention, said chewing gum substance comprises at least one elastomer plasticizer.

In an embodiment of the invention, said chewing gum substance comprises at least one biodegradable polymer.

In an embodiment of the invention, the center-filled chewing gum product being a compressed center-filled chewing gum product.

By a “compressed center-filled chewing gum product” is herein meant a compressed center-filled chewing gum tablet obtained by a compression technique, i.e. by application of tabletting equipment.

The present invention further relates to use of a center-filled chewing gum product as described above for cleaning of teeth.

The present invention further relates to use of a center-filled chewing gum product as described above as a replacement for a toothbrush.

Cleaning of teeth by using the center-filled chewing gum product according to the invention may advantageously result in a cleaning effect corresponding to the cleaning effect obtained by use of a conventional toothbrush with toothpaste.

The present invention further relates to use of at least one anti-plaque agent, at least one anti-gingivitis agent, at least one re-mineralization agent, at least one whitening agent, at least one fresh-breath agent, and optionally at least one anti-calculus agent in a center-filled chewing gum product, for preventing the risk of tooth and gingiva damage inherent in tooth brushing by maintaining such oral hygiene by chewing the chewing gum product that need for brushing is substantially unneeded.

The present invention further relates to a method of obtaining a center-filled chewing gum product as described above comprising the steps of
extruding chewing gum substance and toothpaste as a rope in which said chewing gum substance is enclosing a center-filling comprising said toothpaste,
cutting said rope to individual rope-pieces, each having two ends, and
closing both of said two ends of said rope pieces.

The present invention further relates to a method of obtaining a center-filled chewing gum product as described above comprising the steps of
providing a granulated chewing gum substance,
providing a center-filling material,
transferring a first part of said granulated chewing gum substance to a tabletting machine comprising a piston comprising a centered pivot and a further piston with flat surface,
tabletting said first part of said granulated chewing gum substance by said piston comprising a centered pivot leaving said first part of said granulated chewing gum substance as a compressed chewing gum substance comprising a centered hole,
transferring said center-filling material to said centered hole,
transferring a second part of said granulated chewing gum substance onto said compressed first part of said granulated chewing gum substance and onto said center-filling material,
tabletting said second part of said granulated chewing gum substance by said further piston with flat surface.

The present invention further relates to a chewing gum substance comprising anti-plaque agent and being configured to enclose a center-filling which comprises toothpaste.

The present invention further relates to a packaging for a center-filled chewing gum product as described above, said packaging comprising an indication in writing and/or drawing that the center-filled chewing gum product inside that packaging comprises toothpaste.

### BRIEF DESCRIPTION OF THE DRAWINGS

The invention will be described with reference to the drawings of which

FIG. 1 illustrates the process steps relating to continuous forming of the center-filled chewing gum products according to the present invention,

FIGS. 2A-2E illustrate cross sectional views of extruded chewing gum rope with center-filling,

FIGS. 3A-3H illustrate examples of different shapes of the center-filled chewing gum products according to the present invention, and

FIGS. 4A-4B illustrate examples in principle of chewing gum products with center-filling, chewing gum substance, anti-sticking agent and coating, and

FIGS. 5A-5D illustrate examples in principle of center-filled chewing gum products comprising dotted or striped patterns of the center-filling or of the enclosing chewing gum substance.

### DETAILED DESCRIPTION OF THE INVENTION

When preparing center-filled chewing gum products according to the invention, many aspects have to be taken into consideration, especially in order to provide the dental care effects according to the invention. Here below is given a detailed description of the components and processes involved, when manufacturing center-filled chewing gum products according to the present invention.

The center-filled chewing gum product according to the present invention comprises a chewing gum substance enclosing a center-filling, which comprises toothpaste. Furthermore, the product comprises functional agents including at least one anti-plaque agent.

The center-filling according to the invention is preferably in the form of a paste or a gel facilitating a particular advantage by counteracting problems associated with an entirely liquid center-fill. The terms “paste” and “gel” should be understood in a broad sense and include materials, which may be characterized as “paste-like” or “gel-like”. The term paste may typically be understood as a rather smooth and viscous mixture, which may usually comprise a suspension of granular material in a background fluid, whereas a gel may typically be characterized as a colloid or colloidal suspension in which the disperse phase has combined with the dispersion medium to produce a semisolid material.

The center-filling according to the invention may comprise both dissolved and suspended components, and preferably the center-filling comprises functional agents. Targeting the problems related to liquid-fillings, a paste-like consistency may according to the invention facilitate an immobilization of the center-fill with regard to the surrounding chewing gum substance. Specifically, problems related to reactivity and storage ability of the functional agents in the center-fill and problems with leakage from the chewing gum product or dissolving of chewing gum substance may be counteracted by a applying a center-filling in the form of a paste or a gel. The enduring quality of the product may in this way be prolonged. Concurrently, it is preferred according to the invention that the preferably gel- or paste-like center-filling contains an amount of liquid. The liquid content may facilitate a ready-for-use product, as the functional agents are already dispersed in a solution when the product is taken into the mouth and thus the functional agents are ready for immediately performing the intended dental care action. For this purpose, it may be preferred that the liquid content includes water as solvent for functional agent, e.g. functional agents in the form of salts.

In general, the composition of the chewing gum substance according to the invention typically comprises a water-soluble bulk portion, a water-insoluble chewable gum base portion, and flavoring agents. The water-soluble portion dissipates with a portion of the flavoring agent over a period of time during chewing. The gum base portion is retained in the mouth throughout the chew. The term “chewing gum” refers to both a chewing and bubble type gum in its general sense.

Unless otherwise stated, percentages given herein are percentages by weight.

In addition to the water insoluble gum base portion, the chewing gum substance applied according to the present invention typically includes one or more flavoring agents and a water soluble bulk portion, which may include bulk sweeteners, high intensity sweeteners, flavoring agents, softeners, emulsifiers, colors, acidulants, fillers, antioxidants, and other components that provide desired attributes.

Turning first to the water-insoluble gum base part, which forms the masticatory part of the final chewing gum substance, and which imparts the chew characteristics to the final product. The gum base typically defines the release profile of flavors, and sweeteners and plays a significant role in the gum product.

The water-insoluble portion of the gum typically may contain any combination of elastomers, vinyl polymers, elastomer plasticizers, waxes, softeners, fillers and other optional ingredients such as colorants and antioxidants. The gum base portion may constitute about 5 to 95 percent by weight of the chewing gum substance, more commonly, the gum base comprises about 10 to 50 percent of the chewing gum substance.

The composition of gum base formulations can vary substantially depending on the particular product to be prepared and on the desired masticatory and other sensory characteristics of the final product. However, typical ranges (weight %) of the above gum base components are: 5 to 50% by weight elastomeric compounds, 5 to 55% by weight elastomer plasticizers, 0 to 40% by weight of waxes, 5 to 35% by weight softener, 0 to 50% by weight filler, and 0 to 5% by weight of miscellaneous ingredients such as antioxidants, colorants, etc.

Elastomers provide the rubbery, cohesive nature to the gum, which varies depending on this ingredient's chemical structure and how it may be compounded with other ingredients. Elastomers suitable for use in the gum base and gum of the present invention may include natural or synthetic types.

The elastomer may be any water-insoluble polymer known in the art, and includes those gum polymers utilized for chewing gum and bubble gum listed in Food and Drug Administration, CFR, Title 21, Section 172,615, as “Masticatory Substances of Natural Vegetable Origin” and “Masticatory Substances, Synthetic”

Useful natural elastomers include natural rubber such as smoked or liquid latex and guayule, natural gums such as jelutong, lechi caspi, perillo, sorva, massaranduba balata, massaranduba chocolate, nispero, rosidinha, chicle, gutta percha, gutta kataiu, niger gutta, tunu, chilte, chiquibul, gutta hang kang.

Useful synthetic elastomers include high molecular weight elastomers such as butadiene-styrene copolymers, polyisobutadiene and isobutylene-isoprene copolymers, low molecular weight elastomers such as polybutene, polybutadiene and polyisobutylene, vinyl polymeric elastomers such as polyvinyl acetate, polyethylene, vinyl copolymeric elastomers such as vinyl acetate/vinyl laurate, vinyl acetate/vinyl stearate, ethylene/vinyl acetate, polyvinyl alcohol or mixtures thereof. In general, some preferred low molecular weight elastomers have molecular weights in the range of 40000-60000 g/mole (Mw), while some preferred high molecular weight elastomers cover the range of 250000-450000 g/mole (Mw).

Butadiene-styrene type elastomers, or SBR as they may be called, typically are copolymers of from about 20:80 to 60:40 styrene:butadiene monomers. The ratio of these monomers affects the elasticity of the SBR as evaluated by mooney viscosity. As the styrene:butadiene ratio decreases, the mooney viscosity decreases.

The structure of SBR typically consists of straight chain 1,3-butadiene copolymerized with phenylethylene (styrene) and provides the non-linear molecular nature of these elastomers. The average molecular weight of SBR is <600000 g/mole.

Isobutylene-isoprene type elastomers, or butyl as they may be called, have molar percent levels of isoprene ranging from 0.2 to 4.0. Similar to SBR, as the isoprene:isobutylene ratio decreases, so does the elasticity, measured by mooney viscosity.

The structure of butyl rubber typically consists of branched 2-methyl-1,3-butadiene (isoprene) copolymerized with branched 2-methylpropene (isobutylene), and, as with SBR, this type of structure is non-linear in nature. The average molecular weight of SBR is in the range from 150000 g/mole to 1000000 g/mole.

Polyisobutylene, or PIB as they may be called, type elastomers are polymers of 2-methylpropene and, as with SBR and butyl, are non-linear in nature. The low molecular weight elastomers provide soft chew characteristics to the gum base and still provide the elastic qualities, as do the other elastomers. Average molecular weights may range from about 30000 to 120000 g/mole and the penetration may range from about 4 millimeters to 20 millimeters. The higher the penetration, the softer the PIB. Similar to the SBR and butyl, the high molecular weight PIB elastomers provide elasticity the gum, and their average molecular weight may range from 120000 to 1000000 g/mole.

Polybutenes range in average molecular weight from about 5000 g/mole to about 30000 g/mole.

Vinyl polymeric and copolymeric type elastomers provide tack resistance, vary the chew characteristics of gums made from these bases having vinyl polymers and offer hydrophilic properties beneficial to sensory perception of the final gums.

For vinyl copolymeric types, the amount of vinyl laurate, vinyl stearate, or ethylene present in the vinyl laurate/vinyl acetate (VLNA), vinyl stearate/vinyl acetate (VSNA), or ethylene/vinyl acetate (EVA) copolymers respectively typically ranges from about 10 to about 60 percent by weight of the copolymer. Average molecular weights of these polymers may range from about 2000 g/mole to about 100000 g/mole.

The vinyl polymers such as polyvinyl alcohol and polyvinyl acetate may have an average molecular weight from about 7000 g/mole to about 65000 g/mole. Polymers of vinyl acetate (PVA), are branched in nature. The degree of branching is increased when vinyl acetate monomers are copolymerized with vinyl laurate, vinyl stearate, ethylene and the like. The higher the degree of branching, the higher the compatibility when blended or compounded with normal-alkanic and iso-alkanic type waxes. The desired consistency of the gum base and chewing gum substance for the filled chewing gum products of the present invention may be obtained by combining high and low molecular weight PVA in the gum base. In this case, low molecular weight PVA covers 7000-17000 g/mole (Mw), while high molecular weight PVA covers 40000-60000 g/mole (Mw). Alternatively, only one PVA-polymer may be applied in the gum base having an intermediate molecular weight such as 20000-35000 g/mole.

It is e.g. common in the industry to combine in a gum base a synthetic elastomer having a high molecular weight and a low-molecular-weight elastomer. Presently preferred combinations of synthetic elastomers include, but are not limited to, polyisobutylene and styrene-butadiene, polyisobutylene and polyisoprene, polyisobutylene and isobutylene-isoprene copolymer (butyl rubber) and a combination of polyisobutylene, styrene-butadiene copolymer and isobutylene isoprene copolymer, and all of the above individual synthetic polymers in admixture with polyvinyl acetate, vinyl acetate-vinyl laurate copolymers, respectively and mixtures thereof.

Elastomer plasticizers vary the firmness of the gum base. Their specificity on elastomer inter-molecular chain breaking (plasticizing) along with their varying softening points cause varying degrees of finished gum firmness and compatibility when used in base. This may be important when one wants to provide more elastomeric chain exposure to the alkanic chains of the waxes.

The vinyl polymers such as polyvinyl acetate may often imply at least some of the elastomer plasticizing function, and further elastomer plasticizers suitable for use in the present invention include natural resins including natural rosin esters often referred to as ester gums. Such elastomer plasticizers known in the art are methyl, glycerol and pentaerythritol esters of rosins and modified rosins, such as hydrogenated, dimerized and polymerized rosins. Examples are, glycerol ester of wood and gum rosin, glycerol ester of partially hydrogenated wood and gum rosin, glycerol ester of polymerized wood and gum rosin, glycerol ester of partially dimerized wood and gum rosin, glycerol ester of tall oil rosin, pentaerythritol ester of wood and gum rosin, pentaerythritol esters of partially and fully hydrogenated wood and gum rosin, methyl esters of wood and gum rosins and partially and fully hydrogenated methyl esters of wood and gum rosin.

Useful synthetic elastomer plasticizers include terpene resins derived from alpha-pinene, beta-pinene and/or d-limonene.

The elastomer plasticizers used may be of one type or of combinations of more than one type. Typically, the ratios of one to the other are dependent on each respective softening point, the effect on flavor release, and the respective degree of tack they cause to the gum. Ball and ring softening points of the rosin ester types described above may range from about 45° C. to about 120° C. Softening points of the terpene resins may range from about 60° C. to about 130° C.

The chewing gum substance applied in the filled chewing gum piece according to the present invention may comprise biodegradable polymers. If biodegradable polymers are applied, they may constitute an amount of 10 to 100% by weight of the gum base.

In the present context, the term biodegradable polymer refers to chewing gum base polymers which, after dumping the chewing gum, are capable of undergoing a physical, chemical and/or biological degradation whereby the dumped chewing gum waste becomes more readily removable from the site of dumping or is eventually disintegrated to lumps or particles which are no longer recognizable as being chewing gum remnants. The degradation or disintegration of such degradable polymers can be effected or induced by physical factors such as temperature, light, moisture, by chemical factors such as hydrolysis caused by a change in pH or by the action of enzymes capable of degrading the polymers.

Some biodegradable polymers suitable according to the present invention may include polymers obtained by ring-opening polymerization of cyclic esters or carbonates or polyesters obtained by the polymerization of multi-functional alcohols or derivatives thereof with multi-functional carboxylic acids or derivatives thereof.

Regarding the polyesters obtained by ring-opening polymerization of one or more cyclic esters, their monomers may generally include glycolides, lactides, lactones and/or carbonates. As regards the polyesters prepared from alcohol or derivatives thereof and carboxylic acids or derivatives thereof, examples of preferred polyfunctional carboxylic acids or derivatives thereof are either saturated or unsaturated aliphatic or aromatic and contain 2 to 100 carbon atoms and more preferably 4 to 18 carbon atoms. Furthermore, examples of usually preferred polyfunctional alcohols contain 2 to 100 carbon atoms. Examples of biodegradable polyester polymers may be found in WO 2004/028270, hereby incorporated by reference.

In some embodiments of the invention, the gum base comprises wax, and in other embodiments, for example in case of applying certain biodegradable polymers, wax may be avoided. However, petroleum waxes may aid in the curing of the finished gum made from the gum base as well as improve shelf life and texture. Wax crystal size influences the release of flavor. Those waxes high in iso-alkanes have a smaller crystal size than those waxes high in normal-alkanes, especially those with normal-alkanes of carbon numbers less than 30. The smaller crystal size allows slower release of flavor since there is more hindrance of the flavor's escape from this wax versus a wax having larger crystal sizes. The compatibility of gum bases made using normal-alkanic waxes is less when compared to gum bases made with iso-alkanic waxes. Petroleum wax (refined paraffin and microcrystalline wax) and paraffin wax is composed of mainly straight-chained normal-alkanes and branched iso-alkanes. The ratio of normal-alkanes to iso-alkanes varies. The normal-alkanic waxes typically have carbon chain lengths >C-18 but the lengths are not predominantly longer than C-30. The branched and ring structures are located near the end of the chain for those waxes that are predominantly normal-alkanic. The viscosity of normal-alkanic waxes is <10 mm2/s (at 100° C.) and the combined number average molecular weight is <600 g/mole. The iso-alkanic waxes typically have carbon lengths that are predominantly greater than C-30. The branched chains and ring structures are located randomly along the carbon chain in those waxes that are predominantly iso-alkanic. The viscosity of iso-alkanic waxes is greater than 10 mm2/s (at 100° C.) and the combined number average molekylar weight is >600 g/mole. Synthetic waxes are produced by means atypical of petroleum wax production and thus are not considered petroleum wax. The synthetic waxes may include waxes containing branched alkanes and copolymerized with monomers such as but not limited to propylene and polyethylene and Fischer Tropsch type waxes. Polyethylene wax is a synthetic wax containing alkane units of varying lengths having attached thereto ethylene monomers. The natural waxes may include rice bran wax, bees' wax, carnauba wax or candelilla wax. The waxes may be used alone or in any combination.

Softeners may advantageously be added as well in the gum base portion as in the further water-soluble part of the chewing gum substance. As regards the gum base, the selection of softeners has an influence on the softness of the base. Softeners modify the texture, cause the hydrophobic and hydrophilic components of the base to be miscible, and may further plasticize the synthetic elastomers of the gum base. The emulsifiers, which belong to the group of softeners, provide the gum base with water-binding properties, which confer to the gum base a pleasant smooth surface and reduce its adhesive properties.

Softeners suitable for use in the gum base include triglycerides of non-hydrogenated, partially hydrogenated and fully hydrogenated vegetable oils and tallow, cocoa butter and degreased cocoa powder and in addition to these the emulsifiers.

The group of triglycerides include cottonseed, palm, palm kernel, coconut, safflower, rapeseed, sunflower, tallow, soybean, cocoa butter, medium chained triglycerides and the like.

The caproic, caprylic, capric, myristic, lauric and palmitic fatty acids of the triglycerides tend to plasticize the synthetic elastomers more than triglycerides containing predominantly stearic fatty acid

To the group of emulsifiers belong the monoglycerides, diglycerides, acetylated mono and diglycerides, distilled mono- and diglycerides, glycerol monostearate, propylene glycol monostearate, Na-, K-, Mg- and Ca-stearates, glycerol triacetate, fatty acid monoglycerides (e.g. stearic, palmitic, oleic and linoleic acids), lactic acid esters and acetic acid esters of mono- and diglycerides, sugar esters of edible fatty acids also referred to as sucrose polyesters including those disclosed in WO 00/25598, lecithin and hydroxylated lecithin, most of these may contain triglyceride levels less than 2 percent by weight from their manufacturing processing,

The softeners including emulsifiers may be used alone or two or more in combination. Generally, softeners are added to the chewing gum substance to optimize the chewability and mouth feel of the gum.

Softeners also known in the art as plasticizers may constitute about 0.1 to 15% by weight of the chewing gum substance. Further softener examples contemplated by the present invention include glycerine and lecithine. Also, some aqueous sweeteners, e.g. containing sorbitol, hydrogenated starch hydrolysate or corn syrup, may be used as softeners and binding agents in the chewing gum substance.

Fillers used in gum base modify the texture of the gum base and aid in processing. Particle size has an effect on cohesiveness, density and processing characteristics of the gum base and its compounding. The smaller the particle size, the more dense and cohesive the final gum base. Also, by selecting fillers based on their particle size distribution, initial mass compounding may be varied, thus allowing alteration of the compounding characteristics of the initial mass during gum base processing and ultimately the final chew characteristics of gums made from these gum bases. The filler may constitute between about 5 to 60% by weight of the gum base, preferably about 5 to 50% by weight of the gum base.

Fillers suitable for use in the gum base include magnesium and calcium carbonate, ground limestone and silicate types such as magnesium and aluminum silicate, kaolin and clay, aluminum oxide, silicium oxide, talc, as well as titanium oxide, mono-, di- and tricalcium phosphate, sodium sulphate, cellulose polymers such as ethyl, methyl and wood or mixtures thereof.

Talc filler may be used in the gum base and gum of the present invention that may come in contact with or employ acid flavors or provide an acidic environment needed to prevent degradation of an artificial sweetener by reacting with calcium carbonate type fillers. Mean particle size for calcium carbonate and talc fillers typically range from about 0.1 micron to about 15 microns.

The fillers may also include natural organic fibers such as fruit vegetable fibers, grain, rice, cellulose and combinations thereof.

Antioxidants prolong shelf life and storage of gum base and the final chewing gum substance or their respective components including fats and flavor oils.

Antioxidants suitable for use in gum base include butylated hydroxyanisole (BHA), butylated hydroxytoluene (BHT), betacarotenes, tocopherols, acidulants such as Vitamin C, propyl gallate, other synthetic and natural types or mixtures thereof.

Flavorants and colorants impart characteristics or remove or mask undesired characteristics. They may be applied in the gum base and/or in the mixing of the final chewing gum substance.

Further examples of ingredients, which may be added into the chewing gum substance as a part of the final mixing of water-soluble and water-insoluble parts are given here below. The ingredients are divided into the groups of sweeteners, flavors, surfactants, active ingredients, additives, colors, and material for encapsulating e.g. sweeteners, flavors, or active ingredients.

Suitable bulk sweeteners include sugar free and sugarless sweetening components. Bulk sweeteners typically constitute from about 5 to about 95% by weight of the chewing gum, more typically about 20 to about 80% by weight such as 30 to 60% by weight of the gum.

Sorbitol can be used as a non-sugar sweetener. Other useful non-sugar sweeteners include, but are not limited to, other sugar alcohols such as mannitol, xylitol, hydrogenated starch hydrolysates, maltitol, isomaltol, erythritol, lactitol and the like, alone or in combination.

High-intensity artificial sweetening agents can also be used alone or in combination with the above sweeteners. Preferred high-intensity sweeteners include, but are not limited to sucralose, aspartame, salts of acesulfame, alitame, saccharin and its salts, cyclamic acid and its salts, glycyrrhizin, dihydrochalcones, thaumatin, monellin, twin sweet stevioside, neotame and the like, alone or in combination. In order to provide longer lasting sweetness and flavor perception, it may be desirable to encapsulate or otherwise control the release of at least a portion of the artificial sweetener. Techniques such as wet granulation, wax granulation, spray drying, spray chilling, fluid bed coating, coascervation, encapsulation in yeast cells and fiber extrusion may be used to achieve the desired release characteristics. Encapsulation of sweetening agents can also be provided using another chewing gum component such as a resinous compound.

Addition of high-intensity sweeteners may be carried out at the gum base mixing stage or while mixing the final chewing gum substance.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.02 to about 30% by weight, preferably 0.02 to about 8% per weight. Typically, high intensity sweeteners may be applied in a small amount in the range of 0.05 to 1% by weight of the chewing gum substance. When carriers used for encapsulation are included, the usage level of the encapsulated sweetener will be proportionately higher. Combinations of sugar and/or non-sugar sweeteners can be used in the chewing gum formulation processed in accordance with the invention. Additionally, the softener may also provide additional sweetness such as aqueous sugar or alditol solutions.

If a low-calorie gum is desired, a low-caloric bulking agent can be used. Examples of low caloric bulking agents include polydextrose, Raftilose, Raftilin, fructooligosaccharides (NutraFlora®), palatinose oligosaccharides; guar gum hydrolysates (e.g. Sun Fiber®) or indigestible dextrins (e.g. Fibersol®). However, other low-calorie bulking agents can be used.

The chewing gum according to the present invention may contain aroma agents and flavoring agents including natural and synthetic flavorings e.g. in the form of natural vegetable components, essential oils, essences, extracts, powders, including acids and other substances capable of affecting the taste profile. Flavoring agents may in some embodiments of the invention be encapsulated with the encapsulation materials defined elsewhere, below, in this text.

Examples of liquid and powdered flavorings include coconut, coffee, chocolate, cocoa, vanilla, grape fruit, orange, lime, menthol, liquorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

The chewing gum flavor may be a natural flavoring agent, which is freeze-dried, preferably in the form of a powder, slices or pieces or combinations thereof. The particle size may be less than 3 mm, less than 2 mm or more preferred less than 1 mm, calculated as the longest dimension of the particle. The natural flavoring agent may in a form where the particle size is from about 3 μm to 2 mm, such as from 4 μm to 1 mm. Preferred natural flavoring agents include seeds from fruit e.g. from strawberry, blackberry and raspberry.

Various synthetic flavors, such as mixed fruit flavors may also be used in the present chewing gum centers. As indicated above, the aroma agent may be used in quantities smaller than those conventionally used. The aroma agents and/or flavors may be used in the amount from 0.01 to about 30% by weight of the final product depending on the desired intensity of the aroma and/or flavor used. Preferably, the content of aroma/flavor is in the range of 0.2 to 3% by weight of the total chewing gum substance composition.

In an embodiment of the invention, the flavoring agents comprise natural and synthetic flavorings in the form of natural vegetable components, essential oils, essences, extracts, powders, including acids and other substances capable of affecting the taste profile.

Further chewing gum ingredients, which may be included in the chewing gum according to the present invention, include surfactants and/or solubilisers, especially when pharmaceutically or biologically active ingredients are present. As examples of types of surfactants to be used as solubilisers in a chewing gum composition according to the invention, reference is made to H. P. Fiedler, Lexikon der Hilfstoffe für Pharmacie, Kosmetik und Angrenzende Gebiete, pages 63-64 (1981) and the lists of approved food emulsifiers of the individual countries. Anionic, cationic, amphoteric or non-ionic solubilisers can be used. Suitable solubilisers include lecithin, polyoxyethylene stearate, polyoxyethylene sorbitan fatty acid esters, fatty acid salts, mono and diacetyl tartaric acid esters of mono and diglycerides of edible fatty acids, citric acid esters of mono and diglycerides of edible fatty acids, saccharose esters of fatty acids, polyglycerol esters of fatty acids, polyglycerol esters of interesterified castor oil acid (E476), sodium stearoyllatylate, sodium lauryl sulfate and sorbitan esters of fatty acids and polyoxyethylated hydrogenated castor oil (e.g. the product sold under the trade name CREMOPHOR), block copolymers of ethylene oxide and propylene oxide (e.g. products sold under trade names PLURONIC and POLOXAMER), polyoxyethylene fatty alcohol ethers, polyoxyethylene sorbitan fatty acid esters, sorbitan esters of fatty acids and polyoxyethylene stearic acid esters.

Particularly suitable solubilisers are polyoxyethylene stearates, such as for instance polyoxyethylene(8)stearate and polyoxyethylene(40)stearate, the polyoxyethylene sorbitan fatty acid esters sold under the trade name TWEEN, for instance TWEEN 20 (monolaurate), TWEEN 80 (monooleate), TWEEN 40 (monopalmitate), TWEEN 60 (monoste-arate) or TWEEN 65 (tristearate), mono and diacetyl tartaric acid esters of mono and diglycerides of edible fatty acids, citric acid esters of mono and diglycerides of edible fatty acids, sodium stearoyllatylate, sodium laurylsulfate, polyoxyethylated hydrogenated castor oil, blockcopolymers of ethylene oxide and propyleneoxide and polyoxyethylene fatty alcohol ether. The solubiliser may either be a single compound or a combination of several compounds. In the presence of an active ingredient, the chewing gum may preferably also comprise a carrier known in the art.

Emulsifiers, which are used as softeners may include tallow, hydrogenated tallow, hydrogenated and partially hydrogenated vegetable oils, cocoa butter, glycerol monostearate, glycerol triacetate, lechithin, mono-, di- and triglycerides, acetylated monoglycerides, fatty acids (e.g. stearic, palmitic, oleic and linoleic acids), and combinations thereof.

In one embodiment of the invention, the flavor may be used as taste masking in chewing gum comprising active ingredients, which by themselves has undesired taste or which alter the taste of the formulation.

The chewing gum substance may optionally contain usual additives, such as binding agents, acidulants, fillers, coloring agents, preservatives, and antioxidants, for instance butylated hydroxytoluene (BHT), butyl hydroxyanisol (BHA), propylgallate and tocopherols.

Colorants and whiteners may include FD & C-type dyes and lakes and for example fruit and vegetable extracts, titanium dioxide, and combinations thereof. According to the invention, colors are advantageously applied in the chewing gum substance and/or in the center-filling as a means for generating an intended impression of the product by the consumer. In other words, coloring the product of the invention may be a means for associating the center-filled chewing gum product with a dental care product for brushing of teeth. When looking at the product according to the invention, a consumer may associate the center-filled chewing gum product with a certain toothbrush and or toothpaste, and thus obtaining the impression of a toothbrush replacement.

Suitable compounds for colouring the center-filling material and/or the chewing gum substance may include those classified by the United States Food, Drug, and Cosmetic Act (21 C.F.R. 73), according to which act colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (EI 60b), bixin, norbixin, astaxanthin, dehydrated beets (beet powder), beetroot red/betanin (E 162), ultramarine blue, canthaxanthin (E161g), cryptoxanthin (E161c), rubixanthin (E161d), violanxanthin (E161e), rhodoxanthin (E161f), caramel (E150(a-d)), [beta]-apo-8′-carotenal (E160e), [beta]-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (EI 63), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (EIOI), saffron, titanium dioxide, turmeric (EIOO), turmeric oleoresin, amaranth (E123), capsanthin/capsorbin (E160c), lycopene (E160d), and combinations thereof.

In some embodiments of the invention, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (EI 10), ponceau (E124), erythrosine (E127), patent blue V (E131), titanium dioxide (E171), aluminum (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colors can include FD&C aluminum lakes. These consist of the aluminum salts of FD&C dyes extended on an insoluble substrate of alumina hydrate. Additionally, in some embodiments, certified colors can be included as calcium salts.

Materials to be used for encapsulation methods described herein for encapsulation of e.g. sweeteners, flavors and functional agents might e.g. include the encapsulation materials gelatine, wheat protein, soya protein, sodium caseinate, caseine, gum arabic, modified starch, hydrolyzed starches (maltodextrines), alginates, pectin, carregeenan, xanthan gum, locus bean gum, chitosan, bees wax, candelilla wax, carnauba wax, hydrogenated vegetable oils, zein, and combinations thereof.

In an embodiment of the invention, it may be preferred to include a polyol mixture of sorbitol and one or more polyols having a more densely packed crystalline structure. Such polyol of densely packed crystalline structure may advantageously serve as a moisture barrier component. The polyol mixture may in a preferred embodiment include maltitol, which has a greater crystalline density than sorbitol. Other suitable polyols having a greater crystalline density than sorbitol include xylitol and mannitol. The greater the crystalline density of the polyol the better the barrier properties are. Specifically, a polyol of a greater crystalline density results in a structure with fewer pores, which provides less surface area for potential moisture or fluid migration into the chewing gum composition from the environment and from the liquid part of the paste-like center-filling material.

In an embodiment of the invention, the chewing gum substance may include a polyol composition having a water solubility of less than 72% by weight at 25° C.

Manufacturing of the center-filled chewing gum product according to the invention may be conducted by various different procedures. The gum base, basically comprising the water insoluble parts, may be prepared batch wise or by a continuous process. The gum base may be mixed in a batch or during extrusion and may be cooled and stored before use in the chewing gum production, or the gum base may quite directly enter into the production of the final chewing gum substance. Likewise, the chewing gum substance including all the desired chewing gum ingredients may be produced in a batch process as well as a continuous process, and the chewing gum substance may be cooled and stored, or may be used merely immediately in forming the center-filled chewing gum product of the invention. If applying an extrusion process, the gum base and final chewing gum substance may be prepared in an uninterrupted process, but often it may be preferred to apply a pre-produced gum base in the process. The pre-produced gum base may e.g. be in the form of pellets or larger portions of gum base, which are broken down, warmed, re-mixed and mixed with chewing gum ingredients. The center-filling material may be mixed at room temperature or at elevated temperatures and may involve standard procedures for preparation of toothpastes; suitable procedures for toothpaste preparation are disclosed in e.g. U.S. Pat. No. 6,149,894, U.S. Pat. No. 5,376,360, U.S. Pat. No. 4,795,630, U.S. Pat. No. 5,876,701, U.S. Pat. No. 6,869,595, US2003147817 and WO00/69401, hereby incorporated be reference. The final center-filling may be manufactured by extrusion processes known in the art, such as e.g. disclosed in U.S. Pat. No. 6,838,098, U.S. Pat. No. 6,284,291, U.S. Pat. No. 6,280,780, U.S. Pat. No. 4,316,915, and U.S. Pat. No. 4,399,154, hereby incorporated by reference. Moreover, the process of producing the center-filled chewing gum product according to the invention may involve a tabletting procedure, i.e. enclosing the center-filling in a compressed tablet.

Whether, the production of gum base, chewing gum substance and final center-filled product is performed in separate steps or as an integrated process is not limiting according to the invention. Also, the procedure by which the chewing gum is filled, is not limiting. However, it is essential according to the present invention that the final center-filled chewing gum product comprises anti-plaque agent and that the product comprises toothpaste and one or more functional agents in the center-filling.

In particular, the content of abrasive in the toothpaste provides advantageous effects of the product as regards emulating the function of toothbrushing and thus providing a feasible replacement for a conventional toothbrush.

Further typical components of the center-filling, toothpaste and functional agents are included in the following description.

The overall center-filling generally comprises toothpaste in an amount of at least 10% by weight, preferably at least 50% by weight, and most preferably at least 80% by weight of the overall center-fill composition. In an embodiment of the invention, the entire center-filling is made of toothpaste.

The typical components of toothpaste according to the invention include at least one liquid, at least one humectant, at least one abrasive, and at least one thickener. The liquid preferably comprises water. Usually, the toothpaste comprises further ingredients such as dispersants, surfactants, flavors, sweeteners, colors and active ingredients. Preferably, the active ingredients of the toothpaste are selected among the functional agents listed herein.

In addition to toothpaste, the center-filling may optionally comprise further bulk sweeteners and high-intensity sweeteners.

Bulk sweeteners may comprise 2% to 95% by weight, preferably 5% to 90% by weight, and most preferably 10% to 80% by weight of the overall center-filling. Preferred examples of bulk sweeteners according to the invention include sorbitol, mannitol, xylitol, hydrogenated starch hydrolysates, maltitol, isomaltol, erythritol, and lactitol

Preferably, high-intensity sweeteners are applied in the center-filling in an amount of 0.01% to 4% by weight, more preferably 0.1% to 1% by weight of the overall center-filling. Suitable examples of high-intensity sweeteners include sucralose, aspartame, salts of acesulfame, alitame, saccharin and its salts, cyclamic acid and its salts, glycyrrhizin, dihydrochalcones, thaumatin, monellin, twin sweet stevioside, neotame, and sucram.

The toothpaste and the overall center-filling applied according to the invention may be recognized as a gel or a paste. Several advantages are obtained according to the invention by providing the center-filling with gel-, or paste-consistency as compared to liquid consistency. According to the invention, the obtained advantages include that a more dimensionally stable product may be obtained. Moreover, the center-filling may form a larger percentage of the product without compromising the dimensional stability and the durability of the product. Furthermore, by immobilising the liquid in a gel or paste, the advantage of reduced leaking problems has been obtained.

Without compromising the gel- or paste-like consistency, the amount of liquid present in the center-filling may comprise in the range of 5% to 95%, preferably 10% to 80%, and most typically 20% to 70% by weight of the center-filling.

Suitable humectants for the toothpaste and center-filling according to the invention include glycerine (i.e. glycerol), sorbitol, maltitol, mannitol, xylitol, lactitol, propylene glycol, polyethylene glycol, polypropylene glycols. Humectants often in mixture with water are referred to as liquid vehicles within the art. Humectants are serving to retain moisture in the toothpaste and overall center-filling and to prevent separation of liquid and solids and they are contributing to the desired texture of the center-filling.

Suitable examples of abrasives include sodium metaphosphate, potassium metaphosphate, sodium hexametaphosphate, tricalcium phosphate, calcium phosphate dehydrate, anhydrous dicalcium phosphate, calcium pyrophosphate, magnesium orthophosphate, trimagnesium phosphate, magnesium carbonate, calcium carbonate, baking soda, aluminum silicate, magnesium silicate, zirconium silicate, titanium dioxide, zinc oxide, hydrated alumina, bentonite, hydrated silica, silica gel or colloidal silica, alkali metal aluminosilicate complexes and alumina. If the abrasives are applied as slurries, the liquid, typically water, part of the slurry may be regarded as a carrier and not as an actual part of the abrasive. The solid part of the abrasive slurry may typically comprise 40% to 90% by weight. The most advantageous amount of abrasive is an amount, which is sufficient, in cooperation with the functional agents applied in the product, to emulate during chewing the cleaning effect from a toothbrush. Preferably, the amount of solid abrasive comprises at least 10% by weight, more preferred at least 15%, and most preferred at least 20% by weight of the center-filling. Depending on the type and particle size of the abrasive applied, the amount of abrasive may be up to about 70% by weight of the center-filling. However, it is crucial according to the invention that abrasives are not added in an amount, which causes abrasive damages to the teeth when the product of the invention is chewed. In an advantageous embodiment of the invention, abrasives are in addition to being part of the center-filling also forming part of the chewing gum substance. The inclusion of abrasives in two parts of the product may facilitate an improved abrasive action. According to the invention, the delivery of abrasives from the product onto the teeth may be realized in two rates, i.e. both rather immediately via the toothpaste and over a prolonged period of chewing via the chewing gum substance.

Abrasives included in the center-filled chewing gum product are acting only on the outer portions of the teeth where they cannot cause an abrasive damaging effect on the teeth because a toothbrush is not applied to push against the teeth. Thus, the advantage is obtained by the center-filled chewing gum product as compared to a toothbrush that a more gentle abrasive action is obtained. It is advantageously obtained that the teeth are cleaned during chewing of the chewing gum product of the invention, while the chewing action can not press down gingiva and cause damage to the vulnerable innermost portions of the teeth.

Inorganic dispersants, which may be used in order to stabilize an aqueous slurry of abrasive powder such as those listed above, include inorganic as well as organic dispersants. Examples of inorganic dispersants include condensed phosphates such as pyrophosphates, tripolyphosphates, trimetaphosphates, tetrametaphosphates, and hexametaphosphates, zinc salts and silicates. Organic dispersants include polycarboxylates such as polyacrylates, polymethacrylates, and polymaleates and polyvinyl alcohol.

Thickeners may also be referred to as thickening agents or binders. Thickeners according to the invention are preferably applied to obtain a gel- or paste-like consistency of the toothpaste and of the overall center-filling. Typical amounts of thickeners in the toothpaste and center-filling according to the invention are from 0.05% to 10%, preferably 0.1% to 5% by weight.

Typical thickeners applied in the center-filling according to the invention include pectins, irish moss, starch, natural and synthetic gums and gum-like materials, carboxylmethylcellulose, sodium carboxymethyl cellulose, hydroxybutylmethyl cellulose, hydroxypropylmethyl cellulose, hydroxyethyl cellulose (e.g. Natrosol), hydroxyethyl carboxymethyl cellulose, cellulose ethers, carrageenan, colloidal silica, alginates such as sodium alginate, natural clays such as bentonite, synthetic inorganic clays, and gums such as gum tragacanth, xanthan gum, karaya gum, gum arabic, guar gum, and Carbopols. The gums applied as thickeners are typically hydratable or gelled with water or with alkanols such as polyhydric alcohols like glycerol and sorbitol.

Examples of surfactants useful in the toothpastes applied in accordance with the present invention include anionic surfactants such as sodium myristylsulfate, sodium N-acylsarcosinates (sodium N-lauroylsarcosinate, sodium N-myristoylsarcosinate, N-acylglutamic acid salts (sodium N-palmitoylglutamate, etc.), and sulfosuccinic acid surfactants (polyoxyethylene alkyl disodium sulfosuccinate, dialkyl sodium sulfosuccinate). Examples of nonionic surfactants usable in accordance with the present invention include sugar fatty acid esters (sucrose fatty acid ester, maltose fatty acid ester, lactose fatty acid ester, etc.), polyoxyethylene alkyl ethers, polyoxyethylene sorbitan fatty acid esters (polyoxyethylene sorbitan mono laurate, polyoxyethylene sorbitan monostearate, etc.), polyoxyethylene fatty acid esters (polyoxyethylene-hardened castor oil, etc.), sorbitan fatty acid esters, fatty acid monoglycerides and polyoxyethylene/polyoxypropylene block copolymers. Examples of amphoteric surfactants usable herein include N-alkyldiaminoethylglycine (N-lauryldiaminoethylglycine, N-myristyldiethylglycine, etc.), N-alkyl-N-carboxymethylammonium betaine, 2-alkyl-1-hydroxyethylimidazoline betaine sodium and lauryldimethylaminoacetic acid betaine.

According to an embodiment of the invention, one or a mixture of the above described surfactants may be applied in the toothpaste in an amount of 0.05% to 70% by weight, preferably 0.05% to 20%, and most typically 0.1% to 10% by weight of the toothpaste composition.

Suitable flavors, which may typically be applied in the toothpaste and in the overall center-filling according to the invention includes menthol, arvensis mint oil, synthetic mint flavors, carvone, eugenol, methyleugenol, methyl salicylate, methyl eugenol, thymol, anethole, limonene, ocimene, n-decyl alcohol, citronellol, .alpha.-terpineol, linalol, ethyllinalol, vanillin, thyme, nutmeg, spearmint oil, peppermint oil, lemon oil, orange oil, sage oil, rosemary oil, cinnamon oil, winter green oil, clove oil and eucalyptus oil. Moreover, generally those flavors listed as suitable in the chewing gum composition of the present invention may as well be applied in the center-filling and in the toothpaste according to the invention. In the toothpaste applied according to the invention, either one or a mixture of two or more flavors may be used in an amount, preferably within 0.1% to 5% by weight, most preferably from 0.5% to 2% by weight of the toothpaste composition. Flavors may generally improve the taste of the product according to the invention, and when incorporated in the center-filling, the initial taste sensation is markedly increased as compared to flavors in the chewing gum composition. According to the invention, flavors in the center-filling are useful to cover the taste when bad-tasting functional agents in the product of the invention.

The toothpaste composition in accordance with the present invention may typically contain an amount of 0.001% to 15% of active ingredients including therapeutic ingredients such as water-insoluble noncationic antibacterial agents such as triclosan, Vitamin E, dl-.alpha.-tocopherol acetate, tocopherol succinate, tocopherol nicotinate, Vitamin A, retinol, alpha carotene, beta carotene, Vitamin B, B1-thyamin, B2-riboflavine, B3-niacine, B5-pantothenic acid, B6-pirydoxine, B7-biotine, B8/B9/Bc-folic acid, B12-cianocobalamine, Vitamin C, ascorbic acid, sodium ascorbate, cationic antibacterial agents such as chlorhexidine hydrochloride, cetylpyridinium chloride, enzymes such as dextranase, amylase, protease, mutanase, lysozyme, herbal extracts/oils including chamomile, myrrh, eugenol, tea tree oil, sage oil, mallow, eucalyptus, melissa, pomegranade, apricot, millefolium extract, tangerine extract, natural ingredients (algae, propolis), anticavity alkali metal agents and monofluorophosphates (sodium monofluorophosphate, potassium monofluorophosphate, etc.), fluorides (sodium fluoride, stannous fluoride, etc.), whitening agents (aluminum oxide, calcium peroxide), debriding agent (sodium bicarbonate), astringent salts (such as zinc), chlorophyll, and preservatives such as butylated hydroxytoluene (BHT), butyl hydroxyanisol (BHA), propylgallate and tocopherols, tooth desensitizing agents such as potassium and stronthium salts, condensed anti-tartar phosphates such as sodium and potassium tetrapyrophosphate. Colouring agents suitable for the toothpaste and center-filling are equivalent to those mentioned as colorants for the chewing gum substance.

In a preferred embodiment of the invention, the center-filling mainly comprises toothpaste and in addition an amount of further sweeteners and flavor and functional agents. The further functional agents as compared to conventional toothpaste are preferably applied according to the invention to improve the functionality of the product according to the invention as a toothbrush replacement. Additional sweeteners and flavors may be desired in order to improve the taste, especially when further functional agents have been applied in the center-filling.

In order to obtain an improved dental care function of the product according to the invention, the center-filling may, in addition to the active ingredients listed above as typical in toothpaste, comprise one or more of the further functional agents, which are suitable according to the invention. Examples of suitable functional agents are listed below.

According to the present invention, one or more functional agents are incorporated in the center-filled chewing gum in order to provide a product possessing the desired dental care properties. Preferably, several functional agents are applied, each contributing to improvement of the chewing gum product as replacement for a toothbrush.

An advantage of locating functional agents in the center-filling may according to the invention involve that the functional agents in the center-filling are facilitating an immediate dental care action, whereas functional agents included in the chewing gum substance are released more slowly over a longer chewing period. The immediate action obtained by incorporating functional agents in the center-filling is in particular important, when the consumer is only applying the product for a rather short time period, i.e. such as less than one minute. Although, it may be recommended to use dental care products for at least 1 or 2 minutes, some consumers may want to finish the dental care procedure faster. Moreover, functional agents in the center-filling may be supplied at once in a higher dose as compared to the functional agents released more slowly from the chewing gum substance.

According to a preferred recommendation for use of the present invention, the chewing period of each piece of center-filled chewing gum product should preferably be longer than 2 minutes and up to 20 minutes. Hereby, the consumer may more fully benefit from functional agents included in the chewing gum substance. For some functional agents, the optimal utilization may be obtained when they are supplied to the teeth gradually during a period, preferably of more than 2 minutes. Such functional agents, for which a prolonged action is desired, may preferably be included in the chewing gum substance in order to be released throughout a chewing period.

In a preferred embodiment of the invention, functional agents are included in both the center-filling and in the chewing gum substance. Hereby, an advantageous “double action” may be obtained, which implies that when a consumer takes the product into the mouth and chews, functional agents are supplied immediately from the center-filling, and during continued chewing functional agents are moreover supplied from the chewing gum substance.

Functional agents according to the invention include anti-plaque agents, whitening agents, fresh-breath agents, anti-gingivitis agents, re-mineralization agents, and anti-calculus agents. Anti-plaque agents include anti-bacterial agents and anti-microbial agents. Several compounds have more than one function, i.e. several compounds may be effective as more than one of the functional agent categories according to the invention.

According to the invention, the center-filled chewing gum product includes at least one anti-plaque agent to provide the desired dental care action when used by a consumer. The anti-plaque agent is essential according to the invention in order to obtain a plaque reducing or inhibiting effect comparable to the effect obtainable from brushing with a toothbrush.

Plaque is defined as a bacteria-containing substance that adheres to the surfaces of the teeth as well as on other surfaces of the oral cavity. Hence, by the term “anti-plaque agents” as used herein is meant any agent, which is able to prevent or inhibit the formation and accumulation of bacterial deposits on the surfaces of the oral cavity or to degrade or remove existing bacterial deposits on the surfaces of the oral cavity.

An essential part of dental care, which may be obtained by application of the center-filled chewing gum product according to the invention, involves fighting the build-up of plaque on teeth and other surfaces in the oral cavity. According to the invention, it is possible to fight the bacteria-containing substances on teeth and other surfaces in the oral cavity by application of the center-filled chewing gum product according to the invention, provided that the product contains at least one anti-plaque agent.

Anti-plaque agents according to the invention, i.e. ingredients, which reduce, remove, or inhibit plaque, are preferably selected from those provided in the following. Anti-plaque agents can include xylitol, maltitol, tricolsan, chlorhexidine, zinc acetate, zinc gluconate, zinc citrate, silver nitrate, copper, limonene, pyridinium chloride, and in some embodiments, additional anti-plaque ingredients can be polyphenols, Green tea and extracts hereof, white tea, red tea, coffee and extracts hereof, thyme, oregano, cranberry and extracts hereof, aronia, blueberry, eucalyptus, eucalyptus extract, polyphenoles extracted from berries and plants, zinc ions, copper ions, iron ions and other metal ions. Furthermore, anti-plaque agents may be selected from delmopinol, xylitol, maltitol, immuglobuline-lysozyme (e.g. Ig-lyt or IG-LY 4023 patent WO 2004/060397 A1). Still other examples can include papaine, krillase, pepsin, trypsin, lysozyme, dextranase, amylase, mutanase, glycomaylase, glucose oxidase and a combination thereof. Further anti-plaque agents include the vegetable extracts which are preferably selected from extracts of Centella asiatica, Malva sylvestris, Melaleuca alternifolia, Commiphora abyssinica (myrrh), Krameria triandi (rhatany), Acacia catechu, Medicago sativa (alfalfa), resins of the genus Styrax, such as Styrax benzoin (benzoin), Matricaria recutita (camomile), Echinacea purpurea (echinacea) and Croton lechleri (dragon's blood). Extracts of these plants, whose activity has been known for some time, are commercially available.

Further typical examples of functional agents that are particularly desirable from considerations of anti-plaque effectiveness, safety and formulation include naficillin, oxacillin, vancomycin, clindamycin, erythromycin, trimethoprim-sulphamethoxazole, rifampin, ciprofloxacin, broad spectrum penicillin, amoxicillin, gentamicin, ceftriazoxone, cefotaxime, chloramphenicol, clavunate, sulbactam, probenecid, doxycycline, spectinomycin, cefixime, penicillin G, minocycline, beta-lactamase inhibitors; meziocillin, piperacillin, aztreonam, norfloxacin, trimethoprim, ceftazidime, dapsone; halogenated diphenyl ethers, e.g. 2′,4,4′-trichloro-2-hydroxy-diphenyl ether (Triclosan), 2,2′-dihydroxy-5,5′-dibromo-diphenyl ether; halogenated salicylanilides, e.g. 4′,5-dibromosalicylanilide, 3,4′,5-trichloro-salicylanilide, 3,4′,5-tribromo-salicylanilide, 2,3,3′,5-tetrachloro-salicylanilide, 3,3,3′,5-tetrachloro-salicylanilide, 3,5-dibromo-3′-trifluoromethyl-salicylanilide, 5-n-octanoyl-3′-trifluoromethyl-salicylanilide, 3,5-dibromo-4′-trifluoromethyl-salicylanilide, 3,5-dibromo-3′-trifluoromethyl-salicylanilide (Fluorophene); benzoic esters, e.g. methyl-p-hydroxybenzoic ester, ethyl-p-hydroxybenzoic ester, propyl-p-hydroxybenzoic ester, butyl-p-hydroxybenzoic ester; halogenated carbanilides, e.g. 3,4,4′-trichlorocarbanilide, 3-trifluoromethyl-4,4′-dichlorocarbanilide, or 3,3,4′-trichlorocarbanilide; phenolic compounds (including phenol and its homologs, mono- and poly-alkyl and aromatic halo-phenol and their homologs), e.g. phenol, 2-methyl-phenol, 3-methyl-phenol, 4-methyl-phenol, 4-ethyl-phenol, 2,4-dimethyl-phenol, 2,5-dimethyl-phenol, 3,4-dimethyl-phenol, 2,6-dimethyl-phenol, 4-n-propyl-phenol, 4-n-butyl-phenol, 4-n-amyl-phenol, 4-tert-amyl-phenol, 4-n-hexyl-phenol, 4-n-heptyl-phenol, 2-methoxy-4-(2-propenyl)-phenol (Eugenol), 2-isopropyl-5-methyl-phenol (Thymol), mono- and poly-alkyl- and aralkyl-halophenols, methyl-p-chlorophenol, ethyl-p-chlorphenol, n-propyl-p-chlorophenol, n-butyl-p-chlorophenol, n-amyl-p-chlorophenol, sec-amyl-p-chlorophenol, n-hexyl-p-chlorophenol, cyclohexyl-p-chlorophenol, n-heptyl-p-chlorophenol, n-octyl-p-chlorophenol, o-chlorophenol, methyl-o-chlorophenol, ethyl-o-chlorophenol, n-propyl-o-chlorophenol, n-butyl-o-chlorophenol, n-amyl-o-chlorophenol, tert-amyl-o-chlorophenol, n-hexyl-o-chlorophenol, n-heptyl-o-chloropenol, p-chlorophenol, o-benzyl-p-chlorophenol, o-benzyl-m-methyl-p-chlorophenol, o-benzyl-m,m-dimethyl-p-chlorophenol, o-phenylethyl-p-chlorophenol, o-phenylethyl-m-methyl-p-chlorophenol, 3-methyl-p-chlorophenol, 3,5-dimethyl-p-chlorophenol, 6-ethyl-3-methyl-p-chlorophenol, 6-n-propyl-3-methyl-p-chlorophenol, 6-iso-propyl-3-methyl-p-chlorophenol, 2-ethyl-3,5-dimethyl-p-chlorophenol, 6-sec-butyl-3-methyl-p-chlorophenol, 2-iso-propyl-3,5-dimethyl-p-chlorophenol, 6-diethylmethyl-3-methyl-p-chlorophenol, 6-iso-propyl-2-ethyl-3-methyl-p-chlorophenol, 2-sec-amyl-3,5-dimethyl-p-chlorophenol, 2-diethyl methyl-3,5-dimethyl-p-chlorophenol, 6-sec-octyl-3-methyl-p-chlorophenol, p-bromophenol, methyl-p-bromophenol, ethyl-p-bromophenol, n-propyl-p-bromophenol, n-butyl-p-bromophenol, n-amyl-p-bromophenol, sec-amyl-p-bromophenol, n-hexyl-p-bromophenol, cyclohexyl-p-bromophenol, o-bromophenol, tert-amyl-o-bromophenol, n-hexyl-o-bromophenol, n-propyl-m,m-dimethyl-o-bromophenol, 2-phenyl-phenol, 4-chloro-2-methyl-phenol, 4-chloro-3-methyl-phenol, 4-chloro-3,5-dimethyl-phenol, 2,4-dichloro-3,5-dimethyl-phenol, 3,4,5,6-tetrabromo-2-methylphenol, 5-methyl-2-pentylphenol, 4-isopropyl-3-methylphenol, 5-chloro-2-hydroxydiphenyl-methane; resorcinol and its derivatives, e.g. resorcinol, methyl-resorcinol, ethyl-resorcinol, n-propyl-resorcinol, n-butyl-resorcinol, n-amyl-resorcinol, n-hexyl-resorcinol, n-heptyl-resorcinol, n-octyl-resorcinol, n-nonyl-resorcinol, phenyl-resorcinol, benzyl-resorcinol, phenylethyl-resorcinol, phenylpropyl-resorcinol, p-chlorobenzyl-resorcinol, 5-chloro-2,4-dihydroxydiphenyl-methane, 4′-chloro-2,4-dihydroxydiphenyl-methane, 5-bromo-2,4-dihydroxydiphenyl-methane, 4″-bromo-2,4-dihydroxydiphenyl-methane; and bisphenolic compounds, e.g. bisphenol A, 2,2′-methylene-bis-(4-chlorophenol), 2,2′-methylene-bis-(3,4,6-trichlorophenol) (hexachlorophene), 2,2′-methylene-bis-(4-chloro-6-bromophenol), bis-(2-hydroxy-3,5-dichlorophenyl)-sulfide, and bis-(2-hydroxy-5-chlorobenzyl)-sulfide.

Illustrative of polyphosphate compounds with plaque-inhibiting properties are dialkali metal and tetraalkali metal pyrophosphate and mixtures thereof in a hydrated or unhydrated form. Illustrative of pyrophosphate salts are Na₂H₂P₂O₇, Na₄P₂O₇ and K₄P₂O₇. Other suitable polyphosphates effective as anti-plaque agents include hydrated or unhydrated alkali metal tripolyphosphates such as Na₅P₃O₁₀ and K₅P₃O₁₀.

Plaque buffers such as urea, calcium lactate, calcium glycerophosphate and strontium polyacrylates, ammonium carbonate and vitamins such as Vitamins A, C and E are also included as anti-plaque agents.

Metal cations can also be used as anti-bacterial and anti-plaque agents. The metal cations are selected from the metals of Group 5 (V, Nb, Ta); Group 6 (Cr, Mo, W); Group 7 (Mn, Tc, Re); Group 8 (Fe, Ru, Os); Group 9 (Co, Rh, Ir); Group 10 (Ni, Pd, Pt); Group 11 (Cu, Ag, Au); Group 12 (Zn, Cd, Hg); Group 14 (Ge, Sn, Pb); Group 16 (Se, Te, PO); and mixtures thereof. Preferably the metal cation is selected from any monovalent or divalent cation selected from the group consisting of zinc, manganese, copper, iron, cobalt, silver, selenium, tin and vanadium; preferably from the group consisting of zinc, manganese, copper, iron, silver, and tin; more preferably from the group consisting of zinc, copper, silver and tin and most preferably from the group consisting of zinc and tin.

Illustrative of zinc compounds with plaque-inhibiting properties are zinc oxide, zinc silicate, zinc acetate, zinc carbonate, zinc phosphate, zinc stannate, zinc tetrafluoroborate, zinc hexafluorosilicate, zinc citrate, zinc benzoate, zinc oxalate, zinc stearate, zinc chloride, zinc sulfate, zinc nitrate, zinc phenolsulfonate, zinc carboxymethylsuccinate, and the like. The zinc compound also can be in the form of a complex, with a complexing reagent such as polyethylenimine or ethylenediamine tetraacetic acid.

The effect of chewing a center-filled chewing gum product according to the invention may preferably correspond to at least 60%, preferably in the vicinity of 100% of the anti-plaque effect obtained from brushing of teeth with a toothbrush.

Whitening agents for use herein may comprise materials that remove or bleach intrinsic or extrinsic stains on or in tooth surfaces. Examples of such whitening agents are peroxides, metal chlorites, perborates, percarbonates, peroxyacids, persulphates, and combinations thereof. Suitable peroxide compounds include hydrogen peroxide, urea peroxide, calcium peroxide, sodium bicarbonate, sodium acid pyrophosphate, sodium tripolyphosphate, sodium hexametaphosphate, carbamide peroxide and mixtures thereof. Suitable metal chlorites include calcium chlorite, barium chlorite, magnesium chlorite, lithium chlorite, sodium chlorite and potassium chlorite. As additional bleaching substances hypochlorite, and chlorine dioxide may be mentioned. A preferred percarbonate is sodium percarbonate. Preferred persulphates are oxones. The content of these substances is dependent on the available oxygen or chlorine. The content of these ingredients in the chewing gum according to the invention is generally in the range from about 0.1% to about 35%, preferably from about 1% to about 25% and most preferably from about 5% to about 10%, by weight of the chewing gum.

In a preferred embodiment the whitening agent is selected from the group consisting of baking soda (NaHCO₃), calcium pyrophosphate, calcium carbonate, titandioxid, sodium hexa-metaphosphate, nature identical substitutes thereof, and mixtures thereof.

According to the invention, the effect of chewing a center-filled chewing gum product applied with whitening agents may correspond to at least 50% and preferably in the vicinity of 100% of the whitening effect obtained from brushing of teeth with a toothbrush.

Fresh-breath agents, which are suitable according to the invention, include essential oils, extracts and natural substances as well as various aldehydes, alcohols and similar materials.

In some embodiments, essential oils, extract and natural substances can include spearmint, peppermint, sassafras, chlorophyll, citral geraniol, cardamom, clove, cranberry, blueberry, Aronia melanocarpa, sage, carvacrol, eucalyptus, seaweed, magnolia bark, thyme, oregano, parsley, marjoram, cinnamon, lemon, lime, green tea, red tea, white tea, grapefruit and orange. Furthermore examples of fresh-breath agents include chemicals such as menthol, carvone, iso-garrigol and anethole.

In addition fresh-breath agents can also include zinc citrate, zinc acetate, zinc fluoride, zinc ammonium sulfate, zinc bromide, zinc iodide, zinc chloride, zinc nitrate, zinc formate copper dioxide, carbon-based materials, enzymes such as laccase, lysozyme and combinations thereof. The zinc compounds may also be present as a complex, with a complexing agent such as polyethylenimine or ethylenediamine tetraacetic acid. In some embodiments, the release of probiotics can be used as fresh-breath agents such as Bacillus coagulans, Bacillus subtilis, Lactobacillus acidophilus.

Other agents for fresh breath may include 5-chloro-2-(2,4-dichlorophenoxy)-phenol, commonly referred to as triclosan, and described in the Merck Index, 11^{th} Edition, (1989), pp 1529 (entry No. 9573) in U.S. Pat. No. 3,506,720, and in European Patent publication No. 0 251 591, phthalic acid and its salts including, but not limited to those disclosed in U.S. Pat. No. 4,994,262, preferably magnesium mono-potassium phthalate, chlorohexidine (Merck Index, No. 2090), alexidine (Merck Index, No. 222), hexetidine (Merck Index, No. 4624), sanguinarine (Merck Index, No. 8320), benzalkonium chloride (Merck Index, No. 1066), salicylanilide (Merck Index, No. 8299), domiphen bromide (Merck Index, No. 3411), cetylpyridinium chloride (CPC) (Merck Index, No. 2024), tetradecylpyridinium chloride (TPC), N-tetradecyl-4-ethylpyridinium chloride (TDEPC), octenifine, delmopinol, octapinol, and other piperidine derivatives, nicin preparations, zinc/stannous ion agents, antibiotics, such as augmentin, amoxicilline, tetracycline, doxycycline, hexadine, minocycline, and metronidazole, and analogues and salts of the above, methyl salicyclate, and mixtures of any of the above.

According to the invention, the effect of chewing a center-filled chewing gum product applied with fresh-breath agents may correspond to at least 100% of the fresh-breath effect obtained from brushing of teeth with a toothbrush.

Examples of preferred anti-gingivitis agents, i.e. ingredients for preventing and/or inhibiting gingivitis, are provided in the following. In some embodiments, anti-gingivitis agents may include, Coenzyme Q10, lysozyme, papein, extracts of sassafras, chlorophyll, citral geraniol, cardamom, clove, cranberry, blueberry, Aronia melanocarpa, sage, carvacrol, eucalyptus, seaweed, magnolia bark, thyme, oregano, parsley, marjoram, cinnamon, lemon, lime, green tea, red tea, white tea, grapefruit and orange. Further examples of anti-gingivitis agents are alkali metal, tin fluoride and monofluorophosphates such as sodium and stannous fluoride, sodium monofluorophosphate and mixtures thereof. Further suitable examples of anti-gingivitis agents include mullen leaf, witch hazel, baptisia (wild indigo), marshmallow root (Althea officianales), Potentilla tormentilla, myrrh, agrimony, blood root (sanguinaria), bistort, echinacea, rosemary, ginger, sandalwood, sweet almond, sassafrass, linseed, castor, and products with an antimicrobial effect extracted from milk, e.g. osteopontin. Additionally anti-gingivitis agents may include probiotic bacteria and their perbiotic enhancers e.g. Lactobacillus sp (lactobacillus acidophilus). Bacillus sp. Bifidobacterium sp. (bifidobacterium bifidum) combined with fructose or oligo-fructose.

According to the invention, the effect of chewing a center-filled chewing gum product applied with anti-gingivitis agents may correspond to at least 65% and preferably in the vicinity of 100% of the anti-gingivitis effect obtained from brushing of teeth with a toothbrush.

The term “re-mineralization agents” as used herein includes any agent, which is able to assist in build up of the enamel as well as any agent, which is able to inhibit the demineralization of tooth enamel. Examples of suitable re-mineralization agents are provided in the following. In some embodiments, re-mineralization agents can include tetrasodium pyrophosphate, casein glycomacropeptide, calcium peptone-calcium phosphate, casein phosphopeptide, sodium fluoro phosphate and any salt hereof. Preferred re-mineralization agents include fluorine providing compounds. These compounds may be slightly water soluble or may be fully water soluble and are characterized by their ability to release fluoride ions or fluoride containing ions in water. Typical fluorine providing compounds are inorganic fluoride salts such as soluble alkali metal, alkaline earth metal, and heavy metal salts, for example, sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminum monoand difluorophosphate and fluorinated sodium calcium pyrophosphate and any ion from fluoride. Further examples of agents with re-mineralization effects include Polyfluorite™ System, nano-hydroxyapatite, Novamin®, calcium and phosphate-peptides, CPP-ACP, calcium carbonate, calcium monophosphate, calcium diphosphate, dicalcium phosphate, colostrum, and any derivation from milk, which contains either calcium, phosphate, or fluoride ions.

According to the invention, the effect of chewing a center-filled chewing gum product applied with re-mineralization agents may correspond to at least 65% and preferably in the vicinity of 100% of the re-mineralization effect obtained from brushing of teeth with a toothbrush.

Calculus, also known as tartar, may be defined as hardened deposit composed of mineralised plaque and saliva. Hence, by the term “anti-calculus agents” as used herein is meant any agent, which is able to prevent or reduce the formation of hardened deposit composed of mineralised plaque and saliva.

Anti-calculus agents suitable for use in the chewing gum according to the invention include phosphates, pyrophosphates, alkali-metal pyrophosphates, polyphosphates, sodium tri-polyphosphate, phosphonates, polyphosphonates and mixtures of any of these. Pyrophosphates are among the best known for use in dental care products. The pyrophosphate salts useful in the present invention include the di-alkali metal pyrophosphate salts, tetra-alkali metal pyrophosphate salts and mixtures of any of these in their unhydrated as well as hydrated forms are the preferred species. Di-sodium di-hydrogen pyrophosphate (Na₂H₂P₂O₇), tetra-sodium pyrophosphate (N₄P₂O₇), and tetrapotassium pyrophosphate (K₄P₂O₇) and mixtures thereof are specific examples.

Additional suitable anti-calculus agents include polyacrylates and other polycarboxylates, such as those disclosed in U.S. Pat. No. 3,429,963, U.S. Pat. No. 4,304,766, and U.S. Pat. No. 4,661,341, polyepoxysuccinates, such as those disclosed in U.S. Pat. No. 4,846,650, ethylenediaminetetraacetic acid as disclosed in British Patent No. 490,384, nitrilotriacetic acid and related compounds as disclosed in U.S. Pat. No. 3,678,154, polyphosphonates as disclosed in U.S. Pat. No. 3,737,533, U.S. Pat. No. 3,988,443, and U.S. Pat. No. 4,877,603.

According to the invention, the effect of chewing a center-filled chewing gum product applied with anti-calculus agents may correspond to at least 25% of the anti-calculus effect obtained from brushing of teeth with a toothbrush.

According to an embodiment of the invention, a center-filled chewing gum product may typically comprise fresh-breath agents in an amount of 0.5% to 5%, and anti-plaque agents in an amount of 2% to 60%, and anti-gingivitis agents in an amount of 0.5% to 10%, and whitening agents in an amount of 0.5% to 15%, and re-mineralization agents in an amount of 0.01% to 5% by weight of the overall center-filled chewing gum product.

A suitable whitening effect corresponds to at least 65%, more preferred at least 85%, and even more preferred at least 100%, of the whitening effect of daily brushing of teeth with a new toothbrush.

A suitable anti-plaque effect corresponds to at least 65%, more preferred at least 85%, and even more preferred at least 100%, of the anti-plague effect of daily brushing of teeth with a new toothbrush.

A suitable anti-gingivitis effect corresponds to at least 75%, more preferred at least 85%, and even more preferred at least 100%, of the anti-gingivitis effect of daily brushing of teeth with a new toothbrush.

A suitable re-mineralization effect corresponds to at least 75%, more preferred at least 85%, and even more preferred at least 100%, of the re-mineralization effect of daily brushing of teeth with a new toothbrush.

A suitable anti-calculus effect corresponds to at least 50%, more preferred at least 65%, and even more preferred at least 100%, of the anti-calculus effect of daily brushing of teeth with a new toothbrush.

In a preferred embodiment of the use of the center-filled chewing gum product according to the invention, the product is intended for chewing one, two, three, four five or more times per day, and preferably after eating.

Types of individual ingredients for which optional managed release from the chewing gum substance and/or center-filling may be desired, include, but are not limited to sweeteners, flavors, functional agents, effervescing ingredients, emulsifiers, sensates, throat care ingredients, colors, etc. and combinations thereof. Ingredients may be available in different forms such as, for example, liquid form, spray-dried form, or crystalline form. In some embodiments, a delivery system or chewing gum composition may include the same type of ingredient in different forms.

In some embodiments, an ingredient's release is modified such that when a consumer chews the center-filled chewing gum product, he or she may experience an increase in the duration of flavor or sweetness perception and/or the ingredient is released or otherwise made available over a longer period of time. Modified release may be accomplished by any method known in the art, such as by encapsulation. Where modified release is due to encapsulation, this may be accomplished by a variety of means such as by spray coating or extrusion.

Additionally, early and extended release of ingredients from the center-filling and/or chewing gum substance may imply the application of ingredients without modified release (sometimes referred to as “free” ingredients), as well as ingredients with modified release. In some embodiments, a free ingredient may be used to deliver an initial amount of an ingredient (e.g., flavor, cooling agent) or an initial sensation or benefit caused by the ingredient (e.g., flavor, cooling, warming, tingling, breath freshening, etc.). In some embodiments, the same ingredient can be provided with modified release characteristics to provide an additional or delayed amount of the same sensation or benefit. By using both the free ingredient and the ingredient with modified release characteristics, the sensation or benefit due to the ingredient may be provided over a longer period of time and/or perception of the sensation or benefit by a consumer may be improved.

As another example, in some embodiments it may be desirable to provide a sustained release of an ingredient, especially from the chewing gum substance, over time. To accomplish sustained release, the ingredient may be modified to allow for a lower concentration of the ingredient to be released over a longer period of time versus the release of a higher concentration of the ingredient over a shorter period of time. A sustained release of an ingredient may be advantageous in situations when the ingredient has a bitter or other bad taste at the higher concentrations. A sustained release of an ingredient also may be advantageous when release of the ingredient in higher concentrations over a shorter period of time may result in a lesser amount of the ingredient being optimally delivered to the consumer.

In some embodiments, the delay in availability or other release of an ingredient from the center-filling and/or chewing gum substance caused by encapsulation of the ingredient may be based, in whole or in part, by one or more of the following: the type of encapsulating material, the molecular weight of the encapsulating material, the tensile strength of the delivery system containing the ingredient, the hydrophobicity of the encapsulating material, the presence of other materials in the chewing gum product (e.g., tensile strength modifying agents, emulsifiers), the ratio of the amounts of one or more ingredients in the delivery system to the amount of the encapsulating material in the delivery system, the number of layers of encapsulating material, the desired texture, flavor, shelf life, or other characteristic of chewing gum product, the ratio of the encapsulating material to the ingredient being encapsulated, etc. Thus, by changing or managing one or more of these characteristics, release of one or more ingredients from the chewing gum product during consumption can be managed more effectively and/or a more desirable release profile for one or more ingredients in the center-filled chewing gum product may be obtained. This may lead to a more positive sensory or consumer experience during consumption of the chewing gum product.

In some embodiments, one or more ingredients may be encapsulated with an encapsulating material to modify the release profile of the ingredient. In general, partially or completely encapsulating an ingredient used in a center-filled chewing gum product with an encapsulating material may delay release of the ingredient during consumption of the chewing gum product, thereby delaying when the ingredient becomes available inside the consumer's mouth, throat, and/or stomach, available to react or mix with another ingredient, and/or available to provide some sensory experience and/or functional or therapeutic benefit. This can be particularly true when the ingredient is water soluble or at least partially water soluble.

In some embodiments, a material used to encapsulate an ingredient may include water insoluble polymers, co-polymers, or other materials capable of forming a strong matrix, solid coating, or film as a protective barrier with or for the ingredient. In some embodiments, the encapsulating material may completely surround, coat, cover, or enclose an ingredient. In other embodiments, the encapsulating material may only partially surround, coat, cover, or enclose an ingredient. Different encapsulating materials may provide different release rates or release profiles for the encapsulated ingredient. In some embodiments, encapsulating material used in a delivery system may include one or more of the following: polyvinyl acetate, polyethylene, crosslinked polyvinyl pyrrolidone, polymethylmethacrylate, polylactidacid, polyhydroxyalkanoates, ethylcellulose, polyvinyl acetatephthalate, polyethylene glycol esters, methacrylicacid-co-methylmethacrylate, ethylene-vinylacetate (EVA) copolymer, and the like, and combinations thereof.

In some embodiments, an ingredient may be pre-treated prior to encapsulation with an encapsulating material. For example, an ingredient may be coated with a “coating material” that is not miscible with the ingredient or is at least less miscible with the ingredient relative to the ingredient's miscibility with the encapsulating material.

In some embodiments, an encapsulation material may be used to individually encapsulate different ingredients in the same chewing gum product. For example, a delivery system may include aspartame encapsulated by polyvinyl acetate. Another delivery system may include ace-k encapsulated by polyvinyl acetate. Both delivery systems may be used as ingredients in the same center-filling or chewing gum substance. For additional examples, see U.S. Patent Application Ser. No. 60/683,634 entitled “Methods and Delivery Systems for Managing Release of One or More Ingredients in an Edible Composition” and filed May 23, 2005, hereby incorporated by reference.

In some embodiments, different encapsulation materials may be used to individually encapsulate different ingredients used in the same center-filled chewing gum product. For example, a delivery system may include aspartame encapsulated by polyvinyl acetate. Another delivery system may include ace-k encapsulated by EVA. Both delivery systems may be used as ingredients in the same center-filling or chewing gum substance. Examples of encapsulated ingredients using different encapsulating materials can be found in U.S. Patent Application Ser. No. 60/655,894 filed Feb. 25, 2005, and entitled “Process for Manufacturing a Delivery System for Active Components as Part of an Edible Composition,” hereby incorporated by reference.

In some embodiments, encapsulation may be employed to provide barrier protection to or from a component rather than to modify the release of the component. For instance, it often is desirable to limit the exposure of acids to other components in a chewing gum composition. Such acids may be encapsulated to limit their exposure to other components, or alternatively, the other components in the chewing gum composition may be encapsulated to limit their exposure to the acid.

There are many ways to encapsulate one or more ingredients with an encapsulating material. For example, in some embodiments, a sigma blade or Banbury™ type mixer may be used. In other embodiments, an extruder or other type of continuous mixer may be used. In some embodiments, spray coating, spray chilling, absorption, adsorption, inclusion complexing (e.g., creating a flavor/cyclodextrin complex), coacervation, fluidized bed coating, or other process may be used to encapsulate an ingredient with an encapsulating material.

Examples of encapsulation of ingredients can be found in U.S. Patent Application Ser. No. 60/655,894, filed Feb. 25, 2005, and entitled “Process for Manufacturing a Delivery System for Active Components as Part of an Edible Composition”, hereby incorporated by reference. Other examples of encapsulation of ingredients can be found in U.S. patent application Ser. No. 10/955,255 filed Sep. 30, 2004, and entitled “Encapsulated Compositions and Methods of Preparation”, hereby incorporated by reference. Further examples of encapsulation of ingredients can be found in U.S. patent application Ser. No. 10/955,149 filed Sep. 30, 2004, and entitled “Thermally Stable High Tensile Strength Encapsulation Compositions for Actives”, hereby incorporated by reference. Still further examples of encapsulation of ingredients can be found in U.S. patent application Ser. No. 11/052,672 filed Feb. 7, 2005, and entitled “Stable Tooth Whitening Gum with Reactive Components”, hereby incorporated by reference. Further encapsulation techniques and resulting delivery systems may be found in U.S. Pat. Nos. 6,770,308, 6,759,066, 6,692,778, 6,592,912, 6,586,023, 6,555,145, 6,479,071, 6,472,000, 6,444,241, 6,365,209, 6,174,514, 5,693,334, 4,711,784, 4,816,265, and 4,384,004, hereby incorporated by reference.

In some embodiments, a delivery system may be ground to a powdered material with a particular size for use as an ingredient in a chewing gum composition. For example, in some embodiments, an ingredient may be ground to approximately the same particle size of the other chewing gum ingredients so as to create a homogeneous mixture. In some embodiments, the delivery system may be ground to a powdered material with an average particle size such as, for example, about 4 to about 100 mesh or about 8 to about 25 mesh or about 12 to about 20 mesh.

In some embodiments, selection of an encapsulating material for one or more ingredients may be based on tensile strength desired for the resulting delivery system. For example, in some embodiments, a delivery system produces delayed or otherwise controlled release of an ingredient through the use of a pre-selected or otherwise desired tensile strength.

In an embodiment of the invention, the encapsulating material for a delivery system is polyvinyl acetate. A delivery system utilizing polyvinyl acetate may be prepared by melting a sufficient amount of polyvinyl acetate at a temperature of about 65° C. to 120° C. for a short period of time, e.g., five minutes. The melt temperature will depend on the type and tensile strength of the polyvinyl acetate encapsulating material where higher tensile strength materials will generally melt at higher temperatures. Once the encapsulating material is melted, a suitable amount of an ingredient (e.g., high intensity sweetener, functional agent, etc.) is added and blended into the molten mass thoroughly for an additional short period of mixing. The resulting mixture is a semi-solid mass, which is then cooled to obtain a solid, and then ground to a sieve size of from about e.g. 30 to 200 (600 to 75 microns). The tensile strength of the resulting delivery system can readily be tested according to ASTM-D638.

For additional information regarding how tensile strength of a delivery system may be used to create managed release of one or more ingredients, see U.S. patent application Ser. No. 11/083,968 entitled “A Delivery System for Active Components as Part of an Edible Composition Having Preselected Tensile Strength” and filed on Mar. 21, 2005, and U.S. patent application Ser. No. 10/719,298 entitled “A Delivery System for Active Components as Part of an Edible Composition” and filed Nov. 21, 2003, both hereby incorporated by reference.

In an embodiment of the invention, the encapsulating material for a delivery system is a combination of polyvinyl acetate and natural resin. Suitable natural resins include natural rosin esters often referred to as ester gums including methyl, glycerol and pentaerythritol esters of rosins and modified rosins, such as hydrogenated, dimerized and polymerized rosins. Examples are, glycerol ester of wood and gum rosin, glycerol ester of partially hydrogenated wood and gum rosin, glycerol ester of polymerized wood and gum rosin, glycerol ester of partially dimerized wood and gum rosin, glycerol ester of tall oil rosin, pentaerythritol ester of wood and gum rosin, pentaerythritol esters of partially and fully hydrogenated wood and gum rosin, methyl esters of wood and gum rosins and partially and fully hydrogenated methyl esters of wood and gum rosin. As used herein, the term “natural resin” may further refer to terpene resins derived from alpha-pinene, beta-pinene and/or d-limonene.

In some embodiments, the release of one or more ingredients from a delivery system may depend on more than tensile strength. For example, the release of the ingredients may be directly related to the tensile strength of the delivery system and the hydrophobicity (i.e., water resistance) of the encapsulating polymer or other material.

As a more specific example, when a delivery system is used in a chewing gum, moisture may be absorbed in the encapsulated ingredient(s) during mastication and chewing of the chewing gum. This may result in softening of the encapsulating material and releasing of the ingredient(s) during the mastication and chewing of the chewing gum. The softening of the encapsulation material depends on the hydrophobicity of the polymer used as the encapsulation material. In general, the higher the hydrophobicity of the polymer, the longer mastication time is needed for softening the polymer.

In an embodiment of the invention, release of an ingredient from a delivery system can be managed or otherwise controlled by formulating the delivery system based on the hydrophobicity of the encapsulating material, e.g., the polymer, for the ingredient. Using highly hydrophobic polymers, the release times of the ingredient can be increased or delayed. In a similar manner, using encapsulating material that is less hydrophobic, the ingredient can be released more rapidly or earlier. The hydrophobicity of a polymer can be quantified by the relative water-absorption measured according to ASTM D570-98. Thus, by selecting encapsulating material(s) for a delivery system with relatively lower water-absorption properties and adding that to a mixer, the release of the ingredient contained in the produced delivery system can be delayed compared to those encapsulating materials having higher water-absorption properties. When in an embodiment of the invention encapsulated ingredients are applied, the “loading” of an ingredient in a delivery system can impact the release profile of the ingredient from the chewing gum product. Loading refers to the amount of one or more ingredients contained in the delivery system relative to the amount of encapsulating material. More specifically, the ratio of the amount of one or more ingredients in a delivery system to the amount of encapsulating material in the delivery system can impact the release rate of the one or more ingredients. For example, the lower the ratio or loading of the amount of one or more ingredients in a delivery system to the amount of encapsulating material in the delivery system, the longer or more delayed will be the release of the one or more ingredients from the delivery system. The higher the ratio or loading of the amount of one or more ingredients in a delivery system to the amount of encapsulating material in the delivery system, the faster or earlier will be the release of the one or more ingredients from the delivery system. This principle can be further employed to manage the release profiles of the one or more ingredients by using higher loading of ingredients designed to be released early in combination with lower loading of ingredients designed to be released later. In some embodiments, the one or more ingredients can be the same or different.

For additional information regarding the relationship of the ratio of the amount ingredient in a delivery system to the amount of encapsulating material in the delivery system to the release of an ingredient from a delivery system, see U.S. patent application Ser. No. 11/134,371 entitled “A Delivery System For Active Components as Part of and Edible Composition Including a Ratio of Encapsulating Material and Active Component” and filed on May 23, 2005, with the U.S. Patent and Trademark Office, hereby incorporated by reference.

In an embodiment of the invention, the center-filled chewing gum product comprises sensate compounds in the center-filling and/or in the chewing gum substance.

Sensate compounds can include cooling agents, warming agents, tingling agents, effervescent agents, and combinations thereof. A variety of well known cooling agents may be employed. For example, among the useful cooling agents are included xylitol, erythritol, dextrose, sorbitol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N,2,3-trimethyl-2-isopropyl butanamide (WS-23), N-ethyl-p-menthane-3-carboxamide (WS-3), isopulegol, 3-(l-menthoxy)propane-l,2-diol, 3-(l-menthoxy)-2-methylpropane-l,2-diol, p-menthane-2,3-diol, p-menthane-3,8-diol, 6-isopropyl-9-methyl-l,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(l-menthoxy)ethan-l-ol, 3-(l-menthoxy)propan-l-ol, 3-(l-menthoxy)butan-l-ol, 1-menthylacetic acid N-ethylamide, l-menthyl-4-hydroxypentanoate, 1-menthyl-3-hydroxybutyrate, N,2,3-trimethyl-2-(l-methylethyl)-butanamide, n-ethyl-t-2-c-6 nonadienamide, N,N-dimethyl menthyl succinamide, substituted p-menthanes, substituted p-menthane-carboxamides, 2-isopropanyl-5-methylcyclohexanol (from Hisamitsu Pharmaceuticals, hereinafter “isopregol”); menthone glycerol ketals (FEMA 3807, tradename FRESCOLAT® type MGA); 3-l-menthoxypropane-l,2-diol (from Takasago, FEMA 3784); and menthyl lactate; (from Haarman & Reimer, FEMA 3748, tradename FRESCOLAT® type ML), WS-30, WS-14, Eucalyptus extract (p-Mehtha-3,8-Diol), Menthol (its natural or synthetic derivatives), Menthol PG carbonate, Menthol EG carbonate, Menthol glyceryl ether, N-tertbutyl-p-menthane-3-carboxamide, P-menthane-3-carboxylic acid glycerol ester, Methyl-2-isopryl-bicyclo (2.2.1), Heptane-2-carboxamide; and Menthol methyl ether, and menthyl pyrrolidone carboxylate among others. These and other suitable cooling agents are further described in the following U.S. patents, all of which are incorporated herein by reference: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation agent can be provided. One such tingling sensation is provided by adding jambu, oleoresin, or spilanthol. In some embodiments, alkylamides extracted from materials such as jambu or sanshool can be included.

Additionally, in some embodiments, a sensation is created due to effervescence. Such effervescence is created by combining an alkaline material with an acidic material. An effervescing system may be included in the product of the invention, either in the center-filling or in the chewing gum substance, or both. According to the invention, an effervescing system may contribute to the tooth brushing perception by the consumer when chewing the center-filled chewing gum product. Thus, the impression of the center-filled chewing gum product as a replacement for conventional tooth brushing may be improved as a result of the effervescing system.

Suitable alkaline material(s) for the effervescing system include alkali metal carbonates, alkali metal bicarbonates, alkaline earth metal carbonates, alkaline earth metal bicarbonates, and combinations thereof. Suitable edible acid(s) for the effervescing system include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. In some embodiments of the invention, the effervescing system may include one or more other ingredients such as e.g. carbon dioxide, flavour, functional agents, etc. Examples of effervescing systems in chewing gum can be found in U.S. Pat. No. 6,235,318, hereby incorporated by reference. Moreover, examples of “tingling” type sensates can be found in U.S. Pat. No. 6,780,443, hereby incorporated by reference.

Further breath freshening agents, which may be suitable in an embodiment of the invention, can include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments breath freshening agent can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, papain, krillase, amylase, glucose oxidase, and combinations thereof.

Moreover, further surfactants which may be suitable in an embodiment of the invention, can include surfactants such as sodium stearate, sodium ricinoleate, and sodium lauryl sulphate. The surfactants may be detergents which impart to the composition detersive and foaming properties. Suitable examples of surfactants are water-soluble salts of higher fatty acid monoglyceride monosulfates, such as the sodium salt of the monosulfated monoglyceride of hydgrogenated coconut oil fatty acids, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

Moreover, the center-filled chewing gum product may in an embodiment of the invention comprise one or more of the following antibacterial agents in the center-filling and/or in the chewing gum substance: Triclosan, chlorhexidine, zinc citrate, silver nitrate, copper, limonene, and cetyl pyridinium chloride. Moreover, additional and preferred anticaries agents can include fluoride ions or fluorine-providing components such as inorganic fluoride salts. In some embodiments, soluble alkali metal salts, for example, sodium fluoride, potassium fluoride, sodium fluorosilicate, ammonium fluorosilicate, sodium monofluorophosphate, as well as tin fluorides, such as stannous fluoride and stannous chloride can be included. In some embodiments, a fluorine-containing compound having a beneficial dental care effect, e.g., diminution of enamel solubility in acid and protection of the teeth against decay may also be included. Examples thereof include sodium fluoride, stannous fluoride, potassium fluoride, potassium stannous fluoride, sodium hexafluorostannate, stannous chlorofluoride, sodium fluorozirconate, and sodium monofluorophosphate, and also urea may be included.

In an embodiment of the invention, the center-filled chewing gum product may further comprise potentiator ingredients, which may intensify, supplement, modify or enhance the taste and/or aroma perception of other materials without introducing a characteristic taste and/or aroma perception of their own. The suitable potentiator ingredients may be designed to intensify, supplement, modify, or enhance the perception of flavor, sweetness, tartness, umami, kokumi, saltiness and combinations thereof. In some embodiments, examples of suitable potentiator ingredients, also known as taste potentiators include, but are not limited to, neohesperidin dihydrochalcone, chlorogenic acid, alapyridaine, cynarin, miraculin, glupyridaine, pyridinium-betain compounds, glutamates, such as monosodium glutamate and monopotassium glutamate, neotame, thaumatin, tagatose, trehalose, salts, such as sodium chloride, monoammonium glycyrrhizinate, vanilla extract (in ethyl alcohol), sugar acids, potassium chloride, sodium acid sulfate, hydrolyzed vegetable proteins, hydrolyzed animal proteins, yeast extracts, adenosine monophosphate (AMP), glutathione, nucleotides, such as inosine monophosphate, disodium inosinate, xanthosine monophosphate, guanylate monophosphate, alapyridaine (N-(l-carboxyethyl)-6-(hydroxymethyl)pyridinium-3-ol inner salt, sugar beet extract (alcoholic extract), sugarcane leaf essence (alcoholic extract), curculin, strogin, mabinlin, gymnemic acid, 3-hydrobenzoic acid, 2,4-dihydrobenzoic acid, citrus aurantium, vanilla oleoresin, sugarcane leaf essence, maltol, ethyl maltol, vanillin, licorice glycyrrhizinates, compounds that respond to G-protein coupled receptors (T2Rs and TIRs) and taste potentiator compositions that impart kokumi, as disclosed in U.S. Pat. No. 5,679,397, hereby incorporated by reference.

Sweetener potentiators, which are a type of taste potentiator, enhance the taste of sweetness. In some embodiments, exemplary sweetener potentiators include, but are not limited to, monoammonium glycyrrhizinate, licorice glycyrrhizinates, citrus aurantium, alapyridaine, alapyridaine (N-(l-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, tagatose, trehalose, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and TIRs) and combinations thereof.

In some embodiments of the invention, the center-filled chewing gum product may include a barrier layer, which is located as a layer between the chewing gum substance and the center-filling. The barrier layer may include lipids, proteins, carbohydrates, synthetic elastomers and combinations thereof. Synthetic elastomers may include, but are not limited to, styrene-butadiene copolymers (SBR), polyisobutylene, isobutylene-isoprene copolymers, polyethylene, polyvinyl acetate and combinations thereof. The thickness of the barrier layer may range from about 0.1 mm to about 1 cm.

According to preferred embodiments of the invention, the center-filling is preferably a paste or gel. Moreover, the appearance of the center-filling may be described as a solution, a suspension, an emulsion, a semi-solid, a creme or a gel. Usually, the center-filling may comprise both liquid and non-liquid components, and preferably, the center-filling comprises water. The non-liquid components may be solid or gaseous. The barrier layer may reduce or prevent leakage of the liquid parts of the center-filling, i.e. the barrier layer may serve to reduce or prevent undesirable migration of liquid out of the center-filling.

The center-filling may comprise two or more distinct liquids (which may or may not be miscible) in the same or different amounts and having distinct or different characteristics, such as a mixture of aqueous and non-aqueous liquids.

In some embodiments, multiple barrier layers may be included in a product according to the invention. In some embodiments, migration of liquid from the center-filling may be reduced by decreasing the porosity of a barrier layer adjacent to the center-fill. In other embodiments, liquid migration from the center-filling may be reduced by providing a barrier layer with decreased porosity that is not adjacent to the center-fill material. For example, a barrier layer composition with low porosity may be used as an intermediate layer.

In further embodiments, migration from the center-filling into the enclosing chewing gum substance is reduced by providing a barrier layer which is formed from a composition comprising gum base and filler. In particular, a suitable barrier layer having low porosity may be prepared from a composition including at least 50% by weight of a gum base and less than 40% by weight of fillers. In some embodiments, the filler is present in the barrier layer composition in an amount of about 20 to about 40% by weight. Fillers can include, for example, bulking agents (e.g., bulk sweeteners, mineral adjuvants, carriers and extenders), flavors and high intensity sweeteners. Examples of such barrier layer compositions are provided in WO 02/094033 A1, which is incorporated herein by reference.

In further embodiments, it may be desirable to increase the hydrophobicity of a barrier layer relative to the composition of the center-filling. For example, in some embodiments, the center-filling is hydrophilic, such as an aqueous solution or suspension in form of a paste or gel. In such case, it may be desirable to provide a barrier layer formed from a hydrophobic substance to serve as an effective barrier against water and water soluble compounds in the center-filling. In some embodiments, the barrier layer is formed from at least one lipid, such as a fat or wax. Fats may include, for example, hydrogenated oils or saturated fatty acids. Waxes may include, for example, paraffin wax or beeswax.

In some other embodiments, it may be desirable to increase the hydrophilicity of a barrier layer relative to the composition of the center-filling. For example, in some embodiments, the center-filling may include hydrophobic compounds, like lipids, oxygen and certain flavors. In such case, it may be desirable to provide a barrier layer formed from one or more hydrophilic polymers to serve as an effective barrier against these hydrophobic compounds. Examples of suitable hydrophilic barriers include, but are not limited to gluten, milk proteins, gelatin, starch, pectinates and cellulose-ethers.

In further embodiments, a barrier layer against hydrophilic as well as hydrophobic compounds in the center-filling may be provided by application of a combination of hydrophobic and hydrophilic substances. For example, it may be desired to provide a barrier against water and oxygen. In this case, lipids and certain polymers can be combined to form an effective barrier layer. Suitable polymers would include, but are not limited to, proteins and polysaccharides. For example, these polymers may include, but are not limited to, gluten, milk proteins, gelatin, starch, pectinates and cellulose-ethers. Furthermore, a barrier layer formed from a combination of hydrophobic and hydrophilic substances may be an emulsion-based barrier layer, or the combination may be obtained by forming the barrier layer as two layers, one comprised of a hydrophobic lipid layer and the other comprised of a hydrophilic polymer layer. The hydrophobic lipid layer may be oriented toward a water containing center-filling to reduce loss of liquidity, and the hydrophilic layer may be oriented toward the chewing gum substance.

In still further embodiments, a barrier layer may be provided which accepts and stops or slows liquid migration out of the center-filling. For example, in some embodiments, the barrier layer may include at least one gelling hydrocolloid. Hydrocolloids are hydrophilic polymers of vegetable, animal, microbial or synthetic origin that generally contain many hydroxyl groups and may be polyelectrolytes. In the barrier layer, gelling hydrocolloids may be used to control liquid migration from the centerfilling. Examples of gelling hydrocolloids which may be used in the barrier layer include agar, alginate, carrageenan, cellulose ethers, such as hydroxypropylmethyl cellulose and methylcellulose, gelatins, gellan gum, locust bean gum, pectin, starches, xanthan gum, and combinations thereof.

According to the invention, the center-filled chewing gum products may be provided with an outer coating providing a pleasant taste and protecting the filled chewing gum piece.

During manufacture of the center-filled chewing gum product according to the invention, especially when applying an extrusion process, it may be desirable to apply an amount of anti-sticking agent onto the surface of the extruded center-filled chewing gum rope. Hereby, a chewing gum substance having a texture suitable for extruding to a small wall thickness may be applied while problems with stickiness of the relatively soft chewing gum texture are counteracted by the application of anti-sticking agent. Both sticking to machinery and individual chewing gum pieces sticking to each other may hereby be prevented.

Examples of suitable anti-sticking agents include calcium hydroxide, talc, D-mannitol, silicon dioxide, sucrose ester, calcium stearate, zink stearate, magnesium stearate, and other metallic stearates, polyoxyethylene monostearates, silicates, polyethylene glycols, silicium dioxide, fumed silica, stearic acid, calcium carbonate, etc. and mixtures thereof.

According to some preferred embodiments of the invention, the center-filled chewing gum product may be coated by an outer coating surrounding the chewing gum substance.

One advantage of providing the center-filled chewing gum products with an outer coating according to the present invention is that an advantageous protective effect may be obtained including that the chewing gum substance is protected from drying up, and hence protected from cracking of the chewing gum substance with leaking as a result. This protective effect is especially important, as the chewing gum substance applied according to the present invention is sugar free or sugarless. Overall, the coating may provide the center-filled chewing gum products of the present invention with an increased storability.

Various coating materials may be applied, and the coating types may be selected from hard coatings, soft coatings, film coatings, or coatings of any type that is known in the art, or a combination of such coatings. The coating may typically constitute 10 to 50 percent by weight of a coated filled chewing gum piece.

When providing the center-filled chewing gum product with a coating, it is crucial according to the present invention that the coating material and any additives in the coating material are not capable of damaging the teeth of a consumer. It is decisive in choosing the coating materials for a product according to the invention that such coating material is not counter-acting the intended dental care purpose of the center-filled chewing gum products of the present invention.

Hard coatings include sugar free or sugarless coatings. A hard coating has significant protective effects, which are typically desired according to the present invention. In a typical process of providing the center-filled chewing gum products with a protective sugar free or sugarless hard coating, the gum centers are successively treated in suitable coating equipment with a polyol solution including as examples sorbitol, maltitol, mannitol, xylitol, erythritol, lactitol and isomalt or e.g. a mono-di-saccharide including as example trehalose. Depending on the stage of coating reached, the polyol solution may contain other ingredients, e.g. fillers, colors, functional agents etc.

Alternatively a sugar free or sugarless soft coating may be applied, e.g. comprising a syrup of a polyol or a mono-di-saccharide, including as examples sorbitol, maltitol, mannitol, xylitol, erythritol, lactitol, isomalt and trehalose.

Furthermore, a film coating may be applied, which comprises film-forming agents such as cellulose derivative, modified starch, dextrin, gelatine, zein, shellec, gum arabic, vegetable gum, synthetic polymer, etc. or a combination thereof.

A coating according to the present invention may typically comprise at least a sweetening polyol, a high intensity sweetener, and a flavor.

In general, it may in some embodiments of the invention be preferred to include at least one additive component in the coating material. Examples of such additive component include binding agents, moisture-absorbing components, film-forming agents, dispersing agents, anti-sticking components, bulking agents, flavoring agents, coloring agents, lipid components, wax components, acids, and functional agents. However, it is preferred according to the invention that the coating is free of bad-tasting compounds, and therefore most functional agents are not well-suited as components of the coating.

Coating is optional according to the invention. If a coated product is desired, the center-filled chewing gum product according to the invention may have any form, shape or dimension that permits the center-filled chewing gum product to be coated using any conventional coating process.

Generally according to the invention, the outer appearance of the final center-filled chewing gum product may for example be a pellet, a cushion-shaped pellet, a tablet, a chunk, a pastille, a pill, a ball, and a sphere, and typically the weight of the chewing gum product including chewing gum substance and center-filling material may for example be within the range of 0.5 to 15 grams, preferably about 1 to 5 grams.

An example of a manufacturing process for preparing a center-filled chewing gum product according to the present invention is described in the below example 1. However, the manufacturing process is not limiting for the product according to the invention. The preparation of gum base and chewing gum substance may be prepared in any way known in the art, and the way of manufacture may be chosen in accordance with the available production facilities.

The following examples of the invention are non-limiting and only provided for the purpose of explanation.

### Example 1

### Manufacturing of Center-Filled Chewing Gum Products

The manufacturing involves providing a chewing gum substance and a center-filling composition comprising toothpaste, and forming a product in which the chewing gum substance encloses the center-filling.

Manufacturing of the chewing gum substance is usually commenced by the preparation of gum base. Gum bases may be prepared by adding an amount of the elastomer, elastomer plasticizer and filler, and on occasion a vinyl polymer, to a heated (10° C.-120° C.) e.g. sigma blade mixer with a front to rear speed ratio of from about 1.2:1 to about 2:1.

An initial amount of the ingredients is added to the mixing kettle and compounding is commenced. The compounding involves breaking down the elastomer and increasing chain branching. The higher the level of filler the higher is the degree of compounding. The longer the time of compounding and use of lower molecular weight or softening point gum base ingredients, the lower the viscosity and firmness of the final gum base.

Compounding typically begins to be effective once the ingredients have massed together. Anywhere from 15 minutes to 90 minutes may be the typical length of compounding time. Preferably, the time of compounding is from 20 minutes to about 60 minutes. The amount of added elastomer plasticizer depends on the level of elastomer and filler present.

After the initial ingredients have massed homogeneously and compounded for the time desired, the balance of the gum base ingredients are added in a sequential manner until a completely homogeneous molten mass is attained. Typically, any remainder of elastomer, elastomer plasticizer, vinyl polymer and filler, are added within 60 minutes after the initial compounding time. The optional waxes and the softeners are typically added after the elastomer and elastomer plasticizers and during the next 60 minutes. Then the mass is allowed to become homogeneous before dumping.

Typical gum base processing times may vary from about one to about three hours, preferably from about 1½ to 2½ hours, depending on the formulation. The final mass temperature when dumped may be between 70° C. and 130° C., and preferably between 100° C. and 120° C. It should be noted, that the time for mixing may be shorter and the temperature considerably lower when manufacturing the gum base and chewing gum substance on the basis of biodegradable polymers. The temperature applied when mixing biodegradable polymers may typically be within the range of 40 to 80° C., at which temperatures degradation during the manufacturing process may be avoided.

The completed molten mass is emptied from the mixing kettle into coated or lined pans, extruded or cast into any desirable shape and allowed to cool and solidify.

Mixing of the chewing gum substance may be conducted in a continuous process or batch-wise in mixers with strong e.g. horizontally placed Z-shaped arms, which processes the gum base and chewing gum ingredients. The mixers may typically be heated to a temperature of 30 to 100° C., such as e.g. 45° C. The gum base may typically be processed in the mixer for 1 to 20 minutes, e.g. 10 minutes, after which sweetener is added and mixing is continued for 1 to 20 minutes, e.g. 7 minutes. Then the flavours and the remaining components including functional agents are added and mixing is continued in 1 to 10 minutes, e.g. 5 minutes. Alternatively flavours and the remaining components may be added in the beginning of the mixing process, i.e. before the addition of the sweeteners, or the ingredients could be added alternately in portions during the mixing process.

When the mixing is completed, the chewing gum substance is taken out into carts, onto trays or the like. The chewing gum substance may be cooled, e.g. to a temperature of 30 to 45° C., or lower.

Those skilled in the art will recognize that many variations of the above-described procedure may be followed. Thus, the chewing gum including all so-called gum base components and also the further chewing gum ingredients may alternatively be extruded in one single process. Furthermore, the manufacturing of chewing gum substance may be directly continued, without any considerable storage time, in an extruder forming the filled chewing gum rope, from which the filled chewing gum products of the present invention are obtained.

A center-filling material is provided, e.g. by purchasing a commercially available toothpaste, or by performing a mixing procedure involving a batch or continuous process. Mixing may be initiated by addition of liquid comprising humectant (polyol) and water, after which abrasive slurry, dispersant, thickener and additional water, sweetener, flavor and therapeutic ingredients may be added in sequence and between each sequence intimate mixing is performed. Those skilled in the art of toothpaste-preparation will recognize many variations in ingredients and in the procedure of manufacture.

Into the prepared or purchased toothpaste, further bulk sweeteners, high intensity sweeteners and functional agents may be mixed to provide the final center-filling material.

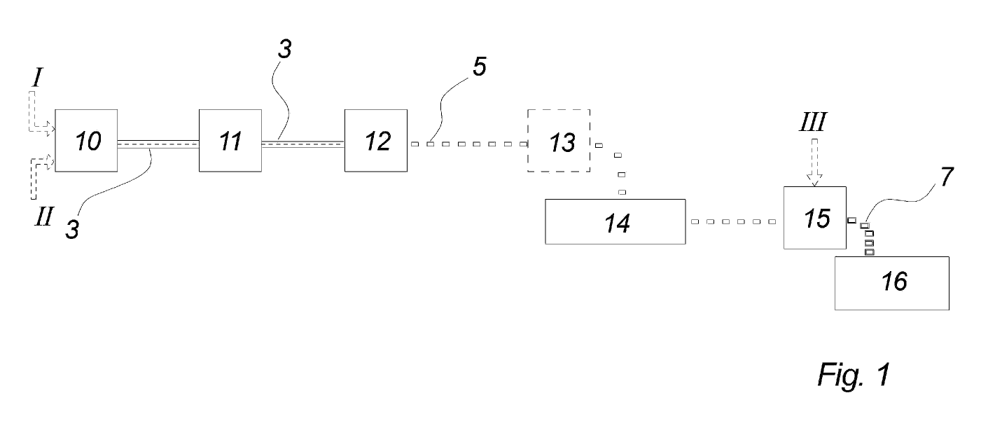

One of several ways of manufacturing the product of the invention, i.e. the center-filled chewing gum products by an extrusion process is described in the following with reference to FIG. 1.

FIG. 1 shows a schematic view of the elements in a continuous process, which may be used to prepare the center-filled chewing gum products according to an embodiment of the present invention.

The illustrated chewing gum manufacturing process involves an extruder 10 feeding a rope sizer 11. The rope sizer 11 is again feeding a tablet forming arrangement 12, which again may, optionally, feed a cooling arrangement 13. An intermediate arrangement 14 connects the past manufacturing process and a following optional coating process involving a coater 15. Finally, the manufacturing process is ended with a storage or packaging arrangement 16.

Initial feeding materials I and II, comprises chewing gum substance I and center-filling material II. The center-filling material II comprises toothpaste and preferably functional agents. The initial feeding materials I and II are introduced into the extruder 10 as indicated by the I-arrow and the II-arrow. A center-filled chewing gum rope 3 is extruded and may be dusted with an anti-sticking agent while continuing to the rope-sizer 11, in which the diameter of the center-filled chewing gum rope 3 is reduced. In the tablet forming arrangement 12, individual pieces of center-filled chewing gum product 5 are formed. The center-filled chewing gum products 5 are uncoated, except from an optional anti-sticking agent. If a coated product is desired, a coating material III is introduced into the coater 15, and the center-filled chewing gum products 5 are coated to form final coated center-filled chewing gum pieces 7, which are stored until they are transferred to a packaging process, or they are packed immediately.

In further details, the process may be explained as follows. Initially, chewing gum substance is mixed, either fresh or as a re-mix of earlier prepared chewing gum pellets, for example in a Z-blade mixer. Alternatively, the mixing may be carried out directly in a continuous process, i.e. the chewing gum substance may be mixed in an extruder. During mixing, the temperature is raised, and a coherent mass of chewing gum substance is obtained.

If the chewing gum substance is mixed in a separate mixer in a batch process, it is transferred to the extruder 10, when the desired texture is achieved. If the chewing gum substance alternatively is made in a continuous process, this mixing may be performed by the illustrated extruder 10 in a known manner.

The extrusion of a chewing gum rope 3 may be carried out at a temperature in the range of 30-90° C., for example the extruder 10 may be heated to a temperature of 35-50° C., and the extruder 10 extrudes a rope of chewing gum substance, while injecting the center-filling material. The material introduced as center-filling II may preferably appear as a liquid, a gel or a paste. The center-filling material comprises toothpaste known in the art and may in addition to toothpaste comprise further sweetener and flavor and further functional agents. When cooled to normal storage temperature such as 20° C., the consistency of the center-filling material is like a gel or paste.

In an embodiment of the invention, it may be advantageous that the chewing gum substance, which is supplied into the extruder, has been waiting, or resting in about at least 15 minutes since mixing or re-mixing before it is extruded by the extruder 10.

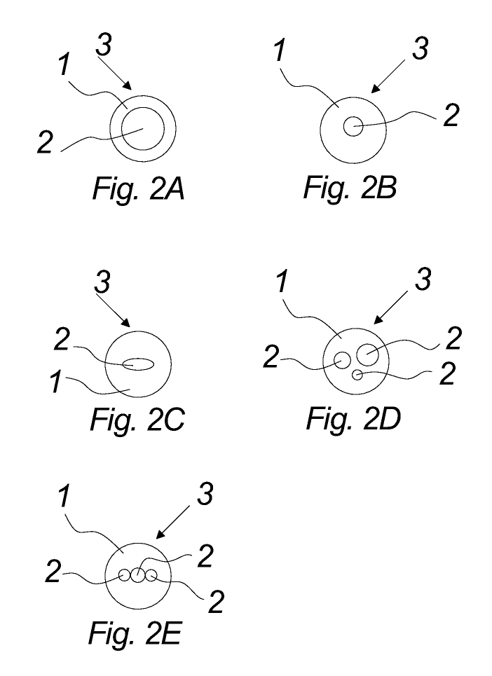

FIGS. 2A-2E illustrates cross-sections of different examples of extruded, filled chewing gum ropes 3. Chewing gum substance 1 encapsulates or encloses a center-filling material 2, which may appear within the rope in different shapes, either in the center or at a distance from the center. The center-filling 2 may for instance appear as a roughly circular or oval shape, and may as illustrated in FIGS. 2D and 2E be extruded as multiple strings of filling material 2 within the chewing gum rope 3. The strings of filling material 2 may have different colors and the filling material 2 of different strings may have different compositions. Filling material 2 supplied into the rope 3 as differently colored strings may appear as differently colored stripes in the final center-filled chewing gum products. Likewise, the filling material 2 may comprise colored beads or particles, which may be desirable in the final product. The chewing gum substance may be prepared as a translucent formulation, in order for the center-filling to be visible within the enclosing chewing gum substance.

Immediately or shortly after the extrusion and before rope-sizing and/or during rope-sizing, the rope is preferably subjected one or more times to application of an anti-sticking agent, e.g. talc, which may for example be sprinkled or dusted onto the rope surface. In the rope-sizing arrangement 11 rope sizing wheels may continuously reduce the diameter of the rope 3 to the desired diameter in order to form the products as pieces of a certain desired size. The chewing gum rope 3 may be reduced in size to a relatively small diameter and wall thickness surrounding the center-filling. For example if desired, a diameter of 16-19 mm and a wall thickness of 2 mm may be obtained.

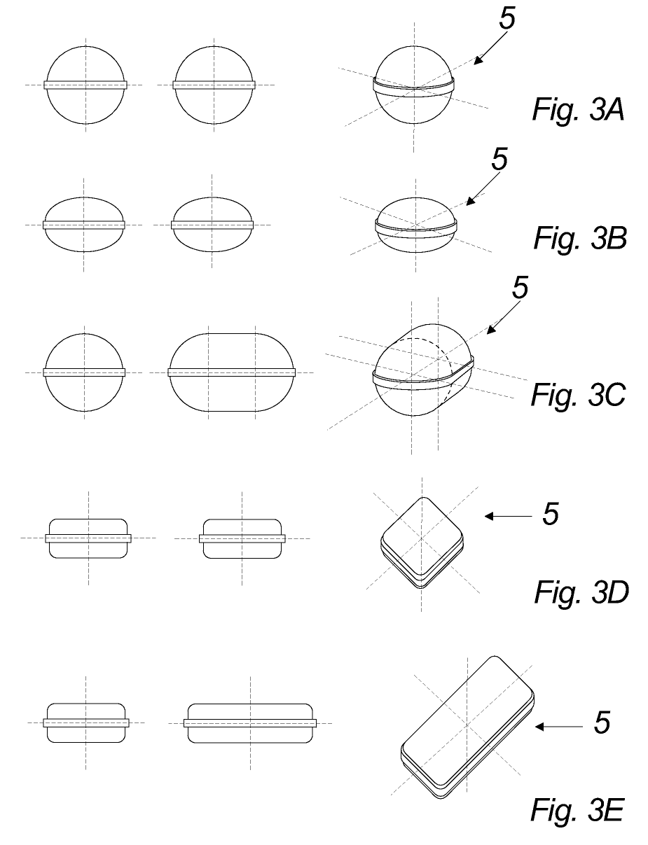

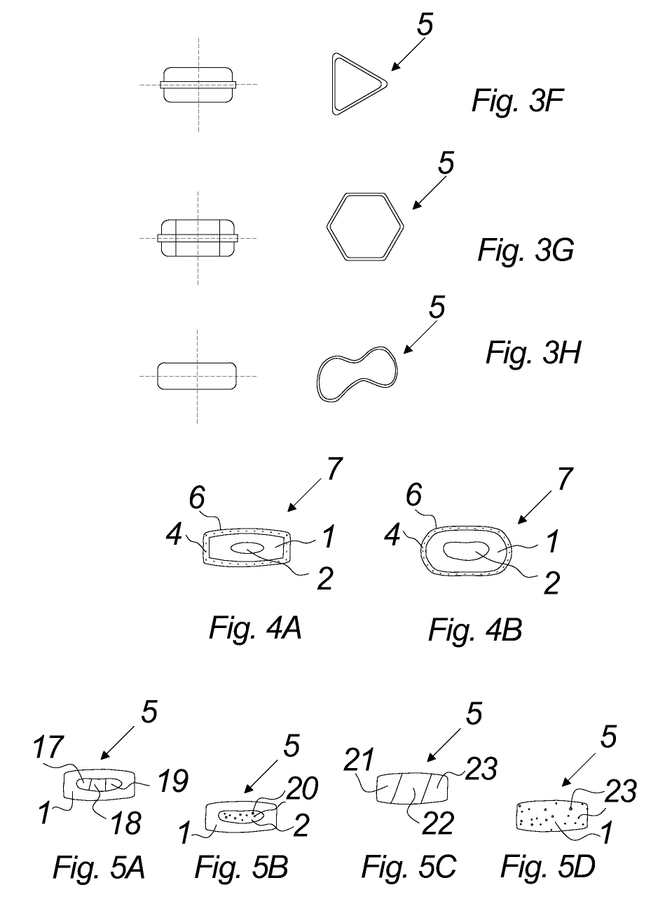

Subsequent to rope sizing, the filled chewing gum rope 3 is fed into a tablet-forming arrangement 12, wherein individual chewing gum products 5 are cut out and formed from the rope. By use of different shapes of e.g. die-cutting tool, different kinds of tablet forms may be produced. Few of many examples hereof are indicated in FIGS. 3A-3H, wherein the center-filled chewing gum products are illustrated in uncoated form, and hence marks, such as a belt around the middle of the chewing gum product, originating from the tablet forming arrangement 12 are visible. The products indicated in FIGS. 3A-3H may have lengths of for example 10 to 50 mm in their longest directions, each of the products 3A-3H considered individually.

Finally, the finished uncoated pieces/products/tablets 5 are cooled, optionally by cooling means 13, either actively, e.g. by a cooling tunnel, or passively as a result of the surrounding temperature in the production area.

To prepare the tablets with a pleasantly sweet or flavored surface, the chewing gum products may be coated to form the finished coated chewing gum products 7, such as illustrated in FIGS. 4A-4B. FIGS. 4A-4B illustrate that the center-filled chewing gum tablets of the present invention comprise a chewing gum substance 1 surrounding a center-filling 2 and being provided with an anti-sticking agent 4 and coated by a coating 6. Since, the anti-sticking agent 4, such as talc, which may be applied on the uncoated tablets during manufacturing, may cause a very undesired mouth feel when chewed directly on, it is important, if anti-sticking agent has been applied, to cover the uncoated tablets with a coating layer 6. The coating layer serves to provide a nice initial taste and sensation in the mouth. Often, the coating layer may be a hard crunchy coating, but other types of coating may be applied as well. Sometimes, a soft- or film-coat may be desired.

The process of extruding, rope-sizing and forming of individual products may in an embodiment of the invention be carried out at temperatures in the range of 30-100° C., preferably in the range of 35-75° C., and typically at about 45-55° C.

FIG. 5A illustrates an example of a center-filled chewing gum product in which the center-filling material is colored to appear as stripes 17, 18, and 19. FIG. 5B illustrates an example of a center-filled chewing gum product in which the center-filling material is dotted. The dots 20 may be colored in various colors, preferably different from the surrounding center-filling material 2.

FIGS. 5A and 5B may be regarded as cross-sectional views. Also, the FIGS. 5A and 5B may be regarded as illustrating that in an embodiment of the invention, a visible center-filling can be obtained by applying a translucent chewing gum substance.

FIG. 5C illustrates an example of a center-filled chewing gum product in which the chewing gum substance is colored to form the stripes 21, 22, and 23. FIG. 5D illustrates an example of a center-filled chewing gum product in which the chewing gum substance is dotted. The dots 24 may be colored in various colors, preferably different from the surrounding chewing gum substance 1.

The dots illustrated in FIGS. 5B and 5D may be formed from colored particles or beads included in the center-filling material and the chewing gum substance, respectively.

### Example 2

### Formulation of Chewing Gum Substance

A gum base is provided and applied in the chewing gum substance given in tables 2 to 4. A suitable gum base may be prepared according to the composition given in table 1:

TABLE 1
Example of a gum base composition, suitable according to the
invention.

| Gum base components | Percentage by weight |
|---|---|
| Elastomer | 19 |
| PVA | 19.9 |
| Natural resins | 20 |
| Filler | 17.5 |
| Softeners | 23.6 |

The gum base may be mixed in a process separate from the mixing of the final chewing gum substance, or the gum base and chewing gum mixing may be carried out uninterrupted in one step. The manufacturing may be by means of extrusion or batch wise, or e.g. as described in Example 1.

It is noted, that softeners and fillers, which in table 1 are included as gum base components, may alternatively be added to the chewing gum as a part of the preparation of chewing gum substance.

In tables 2 and 3, examples are provided of different suitable chewing gum substance compositions comprising functional agents including at least one anti-plaque agent.

TABLE 2
Examples of different chewing gum substance compositions.
Components are given in percentages. Functional agent in the
form of anti-plaque agent is included in each composition along
with one or more further functional agents selected from whitening
agents, anti-gingivitis agents, fresh-breath agents, re-mineralization
agents, and anti-calculus agents.

| sub. | Chew. gum | | | | | | |
|---|---|---|---|---|---|---|---|
| Components | 101 | 102 | 103 | 104 | 105 | 106 | 107 |
| Gum base | 40 | 40 | 40 | 40 | 40 | 40 | 29 |
| Bulk sweetener | 49.5 | 45.5 | 41.5 | 24.5 | 9.5 | 0 | 0 |
| (Sorbitol) | | | | | | | |
| Maltitol syrup | 5 | 5 | 5 | 5 | 5 | 5 | 1 |
| Emulsifier | 0.3 | 0.3 | 0.3 | 0.3 | 0.3 | 0.3 | 0.2 |
| High-intensity | 0.2 | 0.2 | 0.2 | 0.2 | 0.2 | 0.2 | 0.2 |
| sweeteners | | | | | | | |
| Flavour | 3 | 3 | 3 | 3 | 3 | 3 | 3 |
| Anti-plaque agent | 0 | 4 | 8 | 20 | 40 | 49.5 | 64.6 |
| Further functional | 2 | 2 | 2 | 2 | 2 | 2 | 2 |
| agents | | | | | | | |

TABLE 3
Examples of different chewing gum substance compositions.
Components are given in percentages. Functional agent in the
form of anti-plaque agent is included in each composition along
with one or more further functional agents selected from whitening
agents, anti-gingivitis agents, fresh-breath agents, re-mineralization
agents, and anti-calculus agents.

| sub. | Chew. gum | | | | | | |
|---|---|---|---|---|---|---|---|
| Components | 108 | 109 | 110 | 111 | 112 | 113 | 114 |
| Gum base | 40 | 40 | 35 | 30 | 30 | 25 | 24 |
| Bulk sweetener | 31.5 | 27.5 | 26.5 | 24.1 | 21.4 | 19.4 | 12.5 |
| (Sorbitol) | | | | | | | |
| Maltitol syrup | 5 | 5 | 5 | 5 | 5 | 5 | 5 |
| Emulsifier | 0.3 | 0.3 | 0.3 | 0.3 | 0.3 | 0.3 | 0.2 |
| High-intensity | 0.2 | 0.2 | 0.2 | 0.2 | 0.3 | 0.3 | 0.3 |
| sweeteners | | | | | | | |
| Flavour | 3 | 3 | 3 | 3 | 3 | 3 | 3 |
| Anti-plaque agent | 20 | 20 | 20 | 20 | 20 | 20 | 20 |
| Further functional | 0 | 4 | 10 | 17.4 | 20 | 27 | 35 |
| agents | | | | | | | |

If attempting to apply in an embodiment of the invention a chewing gum substance, which contains no functional agents, it is necessary according to the invention to apply functional agents in the center-filling material. Although, a center-filled chewing gum product according to the invention may be obtained when a center-filling containing functional agents is applied in a chewing gum substance with no content of functional agents, it is however preferred according to the invention, that a content of functional agents is included in the chewing gum substance. The dental care effectiveness of the center-filled chewing gum product is improved when the chewing gum substance comprises functional agents, especially when the chewing gum product is chewed for a longer period, such as 2 minutes or more.

In table 4, an example is included the composition making up the 17.4% of further functional agents in chewing gum substance no. 111.

TABLE 4
Example of a chewing gum substance composition including a
composition of functional agents.

| | Chew. gum sub. |
|---|---|
| Components | 111 |
| Gum base | 40 |
| Bulk sweetener (Sorbitol) | 14.2 |
| Maltitol syrup | 5 |
| Emulsifier | 0.3 |
| High-intensity sweeteners | 0.3 |
| Flavour | 3 |
| Anti-plaque agent | 20 |
| Whitening agents | 9.5 |
| Anti-gingivitis agents | 4 |
| Fresh-breath agents | 1.5 |
| Re-mineralization agents | 2.4 |

### Example 3

### Center-Filling Material

Center-filling materials to be enclosed by the chewing gum substance of example 2 may be mixed according e.g. to the compositions given in table 5, or commercially available toothpastes may be purchased and applied as center-filling material and may in some cases be supplemented by additional anti-plaque agent. Suitable toothpastes include such brands as Zendium Sensitive, Colgate Sensitive, Crest Pro Health, etc. In table 5, examples are provided of different toothpaste compositions for center-filling of the center-filled chewing gum products of the invention.

TABLE 5
Examples of different center-filling compositions. Components are
given in percentages. Functional agent in the form of anti-plaque
agent is included in each composition along with one or more
further functional agents selected from whitening agents, anti-gingivitis
agents, fresh-breath agents, re-mineralization agents, and anti-calculus
agents.

| comp. | Center-filling | | | | | | |
|---|---|---|---|---|---|---|---|
| Components | A | B | C | D | E | F | G |
| Humectant | 54.99 | 49.99 | 44.99 | 40 | 30 | 7.49 | 0 |
| Water | 20 | 15 | 12 | 7 | 12 | 5 | 12.5 |
| Thickener | 10 | 10 | 10 | 10 | 1 | 1 | 10 |
| Surfactant | 0.5 | 0.5 | 0.5 | 0.5 | 0.5 | 0 | 0 |
| Abrasive | 10 | 10 | 15 | 20 | 26 | 46 | 10 |
| Flavour | 2.5 | 2.5 | 2.5 | 2.5 | 2.5 | 2.5 | 2.5 |
| Colour | 0.01 | 0.01 | 0.01 | 0 | 0 | 0.01 | 0 |
| Anti-plaque agent | 0 | 10 | 5 | 10 | 0 | 30 | 60 |
| Further functional | 2 | 2 | 10 | 10 | 20 | 0 | 5 |
| agents | | | | | | | |

Either of the chewing gum substance formulations given in tables 2, 3, and 4 may be applied to enclose either of the center-filling formulations given in table 5—except that center-fillings A and E should not be applied within chewing gum substance no. 101.

### Example 4

### Center-Filled Chewing Gum Product Formulations

Suitable examples of formulations of chewing gum products according to the invention are provided in table 6.

TABLE 6
Formulation-examples of center-filled chewing gum products.
Percentages are by weight of the chewing gum substance
or the center-filling, respectively.

| | Center-filled chewing gum products | |
|---|---|---|
| | Ex. 4a | Ex. 4b |
| | % by weight | % by weight |
| Chewing gum substance | | |
| Gum base | 40 | 40 |
| Maltitol syrup | 2 | 2 |
| Mint flavour | 2 | 2 |
| Sucralose | 0.3 | 0.3 |
| Acesulfame-K | 0.15 | 0.15 |
| Xylitol | 55.15 | 54.75 |
| Zn acetate | 0.4 | — |
| Center-filling | | |
| Maltitol syrup | 40 | 34 |
| Carboxy methyl cellulose (CMC) | 1 | 1 |
| Calcium pyrophosphate | 30 | 30 |
| Mint flavour | 2.5 | 2.5 |
| Cranberry extract | 26.1 | 26.1 |
| Sodium fluoride | 0.4 | 0.4 |
| Osteopontin | — | 1 |
| Green tea extract | — | 5 |

It has been found that the center-filled toothpaste-comprising chewing gum products comprising at least one functional anti-plaque agent according to the invention are effective as dental care products for at least partly substituting conventional brushing with a toothbrush.

1. A center-filled chewing gum product comprising functional agents and a chewing gum substance enclosing a center-filling, wherein
said center-filling comprises toothpaste and
said functional agents comprise at least one anti-plaque agent.

2. The center-filled chewing gum product according to claim 1, wherein said functional agents further comprise one or more agents selected from the group consisting of whitening agents, fresh-breath agents, anti-gingivitis agents, re-mineralization agents, and any combination thereof.

3. The center-filled chewing gum product according to claim 2, wherein said functional agents comprise at least one whitening agent, at least one fresh-breath agent, at least one anti-gingivitis agent, and at least one re-mineralization agent.

4. The center-filled chewing gum product according to claim 1, wherein said functional agents further comprise at least one anti-calculus agent.

5. The center-filled chewing gum product according to claim 1, wherein said center-filled chewing gum product comprises said functional agents in an amount of 20% to 55% by weight.

6. The center-filled chewing gum product according to claim 1, wherein said center-filling comprises one or more of said functional agents in a total amount of 10% to 90% by weight of the center-filling.

7. The center-filled chewing gum product according to claim 1, wherein said center-filling comprises at least one anti-plaque agent, at least one whitening agent, at least one fresh-breath agent, at least one anti-gingivitis agent, and at least one re-mineralization agent.

8. The center-filled chewing gum product according to claim 1, wherein at least one of said functional agents is comprised in said toothpaste.

9. The center-filled chewing gum product according to claim 1, wherein said toothpaste comprises at least one anti-plaque agent, at least one whitening agent, at least one fresh-breath agent, at least one anti-gingivitis agent, and at least one re-mineralization agent.

10. The center-filled chewing gum product according to claim 1, wherein said toothpaste constitutes 90% to 100% of said center-filling.

11. The center-filled chewing gum product according to claim 1, wherein said toothpaste constitutes said center-filling.

12. The center-filled chewing gum product according to claim 1, wherein said chewing gum substance comprises one or more of said functional agents in a total amount of 5% to 90% by weight of the chewing gum substance.

13. The center-filled chewing gum product according to claim 2, wherein said chewing gum substance comprises whitening agent in an amount of 1% to 15% by weight of the chewing gum substance.

14. The center-filled chewing gum product according to claim 2, wherein said chewing gum substance comprises fresh-breath agent in an amount of 1% to 10% by weight of the chewing gum substance.

15. The center-filled chewing gum product according to claim 1, wherein said chewing gum substance comprises anti-plaque agents in an amount of 10% to 60% by weight of the chewing gum substance.

16. The center-filled chewing gum product according to claim 2, wherein said chewing gum substance comprises anti-gingivitis agents in an amount 1% to 15% by weight of the chewing gum substance.

17. The center-filled chewing gum product according to claim 2, wherein said chewing gum substance comprises re-mineralization agents in an amount of 0.1% to 10% by weight of the chewing gum substance.

18. The center-filled chewing gum product according to claim 4, wherein said chewing gum substance comprises anti-calculus agents in an amount of 1% to 15% by weight of the chewing gum substance.

19. The center-filled chewing gum product according to claim 1, wherein said center-filling comprises one or more of said functional agents and said chewing gum substance comprises one or more of said functional agents.

20. The center-filled chewing gum product according to claim 1, wherein said center-filling and said chewing gum substance comprise functional agents of the same kinds.

21. The center-filled chewing gum product according to claim 1, wherein said center-filling and said chewing gum substance comprise functional agents of different kinds.

22. The center-filled chewing gum product according to claim 1, wherein two different functional agents are separated in said center-filled chewing gum product by being located in said center-filling and said chewing gum substance, respectively.

23. The center-filled chewing gum product according to claim 1, wherein said toothpaste comprises at least one liquid, at least one humectant, at least one abrasive, and at least one thickener.

24. The center-filled chewing gum product according to claim 23, wherein said at least one abrasive is selected from the group consisting of sodium metaphosphate, potassium metaphosphate, tricalcium phosphate, calcium phosphate dehydrate, anhydrous dicalcium phosphate, calcium pyrophosphate, magnesium orthophosphate, trimagnesium phosphate, calcium carbonate, baking soda, sodium hexametaphosphate, magnesium carbonate, magnesium silicate, titanium dioxide, zinc oxide, aluminum silicate, zirconium silicate, hydrated alumina, bentonite, hydrated silica, silica gel or colloidal silica, alkali metal aluminosilicate complexes, alumina, and any combination thereof.

25. The center-filled chewing gum product according to claim 23, wherein said at least one humectant is selected from the group consisting of glycerine, sorbitol, maltitol, xylitol, lactitol propylene glycol, polyethylene glycol, mannitol, polypropylene glycols, and mixtures thereof.

26. The center-filled chewing gum product according to claim 23, wherein said at least one thickener is selected from the group consisting of pectin, natural and synthetic gums, carboxylmethyl cellulose, sodium carboxymethyl-cellulose, hydroxyethylcarboxymethylcellulose, carrageenan, gum tragacanth, xanthan gum, guar gum, alginates, bentonite, other natural clays, synthetic inorganic clays, gums gelled with water or alkanols, and hydratable gums, irish moss, starch, hydroxy-butylmethyl cellulose, hydroxypropylmethyl cellulose, hydroxyethyl cellulose, cellulose ethers, colloidal silica, karaya gum, gum arabic, and any combination thereof.

27. The center-filled chewing gum product according to claim 1, wherein said toothpaste comprises ingredients selected from the group consisting of surfactants, therapeutic agents, preservatives, and flavour.

28. The center-filled chewing gum product according to claim 1, wherein said toothpaste comprises a mixture of water and humectant, said mixture comprising 5-92% by weight by weight of said toothpaste.

29. The center-filled chewing gum product according to claim 1, wherein said toothpaste comprises water in an amount of 2-90% by weight.

30. The center-filled chewing gum product according to claim 23, wherein said toothpaste comprises said at least one abrasive in an amount of at least 7% by weight.

31. The center-filled chewing gum product according to claim 23, wherein said at least one thickener comprise 0.05% to 10% by weight of said toothpaste.

32. The center-filled chewing gum product according to claim 1, wherein said toothpaste is edible.

33. The center-filled chewing gum product according to claim 2, wherein said whitening agents are selected from the group consisting of magnesium carbonate, calcium carbonate, calcium pyrophosphate, baking soda, sodium hexa-metaphosphate, magnesium silicate, silica, titanium dioxide, zinc oxide, and mixtures thereof.

34. The center-filled chewing gum product according to claim 2, wherein said fresh-breath agents are selected from the group consisting of chlorohexidine, hexetidine, delmopinol, zinc oxide, zinc silicate, zinc carbonate, zinc acetate, zinc phosphate, zinc stannate, zinc citrate, zinc, zinc oxalate, zinc stearate, zinc chloride, zinc sulfate, zinc nitrate, zinc compounds as a complexes, green tea, red tea, white tea, black tea, thyme, eucalyptus, and mixtures thereof.

35. The center-filled chewing gum product according to claim 1, wherein said anti-plaque agents are selected from the group consisting of 2′,4,4′-trichloro-2-hydroxy-diphenyl ether (Triclosan), phenolic compounds (including phenol and its homologs, mono- and poly-alkyl and aromatic halo-phenol and their homologs), phenol, 2-isopropyl-5-methyl-phenol (Thymol), green tea and extracts thereof, white tea, red tea, coffee and extracts thereof, thyme, oregano, cranberry and extracts thereof, aronia, blueberry, eucalyptus, eucalyptus extract, polyphenoles extracted from berries and plants, zinc ions, copper ions, iron ions and other metal ions, delmopinol, xylitol, maltitol, immuglobuline-lysozyme, and mixtures thereof.

36. The center-filled chewing gum product according to claim 2, wherein said anti-gingivitis agents are selected from the group consisting of lactic acid bacteria (LAB), osteopontin (ONP), immuglobuline-lysozyme, aloe vera, chlor-hexidine, green tea, phenolic compounds (including phenol and its homologs, mono- and poly-alkyl and aromatic halo-phenol and their homologs), polyphenoles extracted from berries and plants, and mixtures thereof.

37. The center-filled chewing gum product according to claim 2, wherein said re-mineralization agents are selected from the group consisting of dicalcium phosphate and any calcium ion thereof, sodium fluoro phosphate and any salt thereof, sodium fluoride and any ion from fluoride, nano-hydroxyapatite, Novamin, calcium and phosphate-peptides, CPP-ACP, and mixtures thereof.

38. The center-filled chewing gum product according to claim 1, wherein said functional agents comprise fluoride salts selected from the group consisting of sodium fluoride, potassium fluoride, sodium fluorosilicate, ammonium fluorosilicate, sodium fluorozirconate, sodium monofluorophosphate, and any combination thereof.

39. The center-filled chewing gum product according to claim 4, wherein said anti-calculus agents are selected from the group consisting of vitamin C, citric acid, acetic acid, and any combination thereof.

40. The center-filled chewing gum product according to claim 1, wherein said functional agents comprise an anti-Streptococcus mutans agent.

41. The center-filled chewing gum product according to claim 2, wherein said center-filling comprises at least a part of said fresh-breath agents.

42. The center-filled chewing gum product according to claim 2, wherein said center-filling comprises whitening agent in an amount of 10% to 50% by weight of the center-filling.

43. The center-filled chewing gum product according to claim 2, wherein said center-filing comprises fresh-breath agent in an amount of 10% to 50% by weight of the center-filling.

44. The center-filled chewing gum product according to claim 1, wherein said center-filing comprises anti-plaque agents in an amount of 10% to 90% by weight of the center-filling.

45. The center-filled chewing gum product according to claim 2, wherein said center-filing comprises anti-gingivitis agents in an amount of 10% to 50% by weight of the center-filling.

46. The center-filled chewing gum product according to claim 2, wherein said center-filing comprises re-mineralization agents in an amount of 1% to 50% by weight of the center-filling.

47. The center-filled chewing gum product according to claim 4, wherein said center-filing comprises anti-calculus agents in an amount of 10% to 50% by weight of the center-filling.

48. The center-filled chewing gum product according to claim 1, wherein said center-filling comprises in the range of 2 to 40% by weight of said center-filled chewing gum product.

49. The center-filled chewing gum product according to claim 1, wherein said center-filling comprises 1% to 95% by weight of liquid.

50. The center-filled chewing gum product according to claim 1, wherein said center-filling comprises a gelling agent.

51. The center-filled chewing gum product according to claim 1, wherein said center-filling comprises starch.

52. The center-filled chewing gum product according to claim 1, wherein said center-filling is at least partially solid at temperatures up to 5° C.

53. The center-filled chewing gum product according to claim 1, wherein said center-filling has a viscosity in the range of 0.6 to 200000 mPa*s as measured at 40° C.

54. The center-filled chewing gum product according to claim 1, wherein said center-filling has a paste-like consistency.

55. The center-filled chewing gum product according to claim 1, wherein said center-filling is in the form of a paste, a gel, or any combination thereof.

56. The center-filled chewing gum product according to claim 1, wherein said center-filling comprises a mixture of solid parts and liquid parts.

57. The center-filled chewing gum product according to claim 1, wherein said center-filling comprises 5% to 80% by weight of solid parts.

58. The center-filled chewing gum product according to claim 1, wherein said center-filling is substantially centered in a geometrical center of said center-filled chewing gum product.

59. The center-filled chewing gum product according to claim 1, wherein said center-filled chewing gum product is sugar free or sugarless.

60. The center-filled chewing gum product according to claim 1, wherein said center-filled chewing gum product comprises at least one bulk sweetener.

61. The center-filled chewing gum product according to claim 1, wherein said center-filled chewing gum product comprises at least one high-intensity sweetener.

62. The center-filled chewing gum product according to claim 1, wherein said center-filled chewing gum product comprises at least one foaming agent

63. The center-filled chewing gum product according to claim 1, wherein said foaming agent is a surfactant.

64. The center-filled chewing gum product according to claim 1, wherein said foaming agent comprises an effervescing system including edible acid and edible alkaline material.

65. The center-filled chewing gum product according to claim 1, wherein said center-filling is colored.

66. The center-filled chewing gum product according to claim 1, wherein said center-filling is colored in one, two, three, four, five, or six different colors.

67. The center-filled chewing gum product according to claim 1, wherein said center-filling is colored in stripes.

68. The center-filled chewing gum product according to claim 1, wherein said center-filling is colored in spots.

69. The center-filled chewing gum product according to claim 1, wherein different functional agents are located in different parts of said center-filling, said parts being in different colors.

70. The center-filled chewing gum product according to claim 1, wherein said chewing gum substance and said center-filling are colored differently.

71. The center-filled chewing gum product according to claim 1, wherein said chewing gum substance is colored in stripes or spots.

72. The center-filled chewing gum product according to claim 1, wherein said chewing gum substance is translucent.

73. The center-filled chewing gum product according to claim 1, wherein said center-filling is translucent.

74. The center-filled chewing gum product according to claim 1, wherein said center-filled chewing gum product comprises flavoring agent.

75. The center-filled chewing gum product according to claim 1, wherein said chewing gum substance and said center-filling comprises different flavoring agents.

76. The center-filled chewing gum product according to claim 1, wherein a barrier layer is located between said center-filling and said chewing gum substance.

77. The center-filled chewing gum product according to claim 76, wherein said barrier layer comprises one or more components selected from the group consisting of lipids, proteins, carbohydrates, synthetic elastomers, polymers, and combinations thereof.

78. The center-filled chewing gum product according to claim 76, wherein said barrier layer comprises one or more components selected from the group consisting of wax, fat, gelling hydrocolloid, agar, alginate, carrageenan, glu-ten, milk proteins, gelatin, starch, gellan gum, locust bean gum, pectin, pectinates, xanthan gum, and cellulose-ethers.

79. The center-filled chewing gum product according to claim 1, wherein one or more of said functional agents are encapsulated by one or more encapsulation agents.

80. The center-filled chewing gum product according to claim 79, wherein said encapsulation agents are selected from the group consisting of polyvinyl acetate, polyethylene, crosslinked polyvinyl pyrrolidone, polymethylmethacrylate, polylactidacid, polyhydroxyalkanoates, ethylcellulose, polyvinyl acetatephthalate, polyethylene glycol esters, methacrylicacid-co-methylmethacrylate, ethylene-vinylacetate (EVA) copolymer, fat, wax, natural resins, polyterpene resins, and combinations thereof.

81. The center-filled chewing gum product according to claim 79, wherein at least one of said encapsulation agents comprises a combination of natural resin and polyvinyl acetate.

82. The center-filled chewing gum product according to claim 79, wherein at least one of said encapsulation agents is included in the chewing gum substance.

83. The center-filled chewing gum product according to claim 79, wherein at least one of said encapsulation agents is included in the center-filling.

84. The center-filled chewing gum product according to claim 1, wherein said center-filled chewing gum product is coated by an outer coating.

85. The center-filled chewing gum product according to claim 84, wherein said outer coating is colored.

86. The center-filled chewing gum product according to claim 1, wherein said chewing gum substance forms a wall enclosing said center-filling in substantially all directions.

87. The center-filled chewing gum product according to claim 86, wherein said wall has an average thickness in the range of about 1 to 5 mm.

88. The center-filled chewing gum product according to claim 1, wherein said center-filled chewing gum product weighs in the range of 0.5 to 10 grams.

89. The center-filled chewing gum product according to claim 1, wherein said chewing gum piece is shaped as a pellet, chunk, stick, cushion, pastille, ball, pill, or sphere.

90. The center-filled chewing gum product according to claim 1, wherein said center-filling and said chewing gum substance have a volume-ratio in the range of 1:1 to 1:10.

91. The center-filled chewing gum product according to claim 1, wherein said center-filling and said chewing gum substance have a weight-ratio in the range of 1:1 to 1:10.

92. The center-filled chewing gum product according to claim 1, wherein said chewing gum substance comprises a gum base constituting 20 to 80% by weight of the chewing gum substance.

93. The center-filled chewing gum product according to claim 1, wherein said chewing gum substance comprises at least one elastomer

94. The center-filled chewing gum product according to claim 1, wherein said chewing gum substance comprises at least one elastomer plasticizer.

95. The center-filled chewing gum product according to claim 1, wherein said chewing gum substance comprises at least one biodegradable polymer.

96. The center-filled chewing gum product according to claim 1, said center-filled chewing gum product being a compressed center-filled chewing gum product.

97. Use of a center-filled chewing gum product according to claim 1 for cleaning of teeth.

98. Use of a center-filled chewing gum product according to claim 1 as a replacement for a toothbrush.

99. Use of at least one anti-plaque agent, at least one anti-gingivitis agent, at least one re-mineralization agent, at least one whitening agent, and at least one fresh-breath agent, in a center-filled chewing gum product, for preventing the risk of tooth and gingiva damage inherent in tooth brushing by maintaining such oral hygiene by chewing the chewing gum product that need for brushing is substantially unneeded.

100. A method of obtaining a center-filled chewing gum product according to claim 1 comprising the steps of
extruding chewing gum substance and toothpaste as a rope in which said chewing gum substance is enclosing a center-filling comprising said toothpaste,
cutting said rope to individual rope-pieces, each having two ends, and
closing both of said two ends of said rope pieces.

101. A method of obtaining a center-filled chewing gum product according to claim 1 comprising the steps of
providing a granulated chewing gum substance,
providing a center-filling material,
transferring a first part of said granulated chewing gum substance to a tabletting machine comprising a piston comprising a centered pivot and a further piston with flat surface,
tabletting said first part of said granulated chewing gum substance by said piston comprising a centered pivot leaving said first part of said granulated chewing gum substance as a compressed chewing gum substance comprising a centered hole,
transferring said center-filling material to said centered hole,
transferring a second part of said granulated chewing gum substance onto said compressed first part of said granulated chewing gum substance and onto said center-filling material,
tabletting said second part of said granulated chewing gum substance by said further piston with flat surface.

102. A chewing gum substance comprising anti-plaque agent and being configured to enclose a center-filling which comprises toothpaste.

103. A packaging for a center-filled chewing gum product according to claim 1, said packaging comprising an indication in at least one of writing and drawing that the center-filled chewing gum product inside that packaging comprises toothpaste.
